(19) United States
(12) Patent Application Publication
(10) Pub. No.: US 2020/0402609 A1
(43) Pub. Date: Dec. 24, 2020

(54) COMPUTER IMPLEMENTED DISCOVERY OF ANTIBODY SIGNATURES

(71) Applicant: CEREDX INC., Morgantown, WV (US)

(72) Inventors: Taura L. Barr, Moorefield, WV (US); Grant O'Connell, Cleveland, OH (US)

(21) Appl. No.: 16/975,055

(22) PCT Filed: Feb. 21, 2019
(86) PCT No.: PCT/US2019/018925

§ 371 Date: Aug. 21, 2020

Related U.S. Application Data
(60) Provisional application No. 62/633,188, filed on Feb. 21, 2018.

(51) Int. Cl. G16B 5/20; G01N 33/68; G16B 25/10

(52) U.S. Cl. CPC G16B 5/20; G01N 2800/2871; G16B 25/10; G01N 33/6896

(57) ABSTRACT

Disclosed herein are computer implemented methods of distinguishing ischemic stroke, hemorrhagic stroke, and stroke mimic by detecting circulating biomarkers in a subject. Also provided herein are systems, kits, and methods for the detecting.

### CROSS-REFERENCE

This application claims the benefit of U.S. Provisional Application No. 62/633,188, filed Feb. 21, 2018, which is incorporated by reference herein in its entirety.

### BACKGROUND

Due to the time-efficacy relationship associated with acute stroke interventions, tools which allow for accurate stroke diagnosis during triage have the potential to streamline care and improve patient outcomes. Confident prehospital recognition of stroke by emergency medical services personnel allows for direct transport to certified stroke centers, which not only saves time, but also affords patients access to advanced treatment options not available at smaller medical facilities. Beyond the initial stroke recognition, further prehospital determinations, such as identification of stroke subtype, allow for advanced notice to be given to the receiving medical center, which can be used to expedite treatment once the patient arrives. In the case of self-admitted patients, the ability to make similar diagnostic determinations at the initial point of emergency department contact is equally beneficial through mechanisms such as expedited stroke team referral. Unfortunately, the tools currently available to clinicians for stroke diagnosis during triage are limited, and early care decisions are based predominantly on the evaluation of overt patient symptoms using rudimentary stroke severity and recognition scales. In view of these limitation, tools for the identification of stroke-associated peripheral blood biomarkers which could be rapidly measured at the point of care are needed.

The peripheral immune system can play a central role in stroke pathology; not only may there be a rapid systemic inflammatory response to the acute injury, but emerging evidence suggests that peripheral immune changes may proceed symptom onset and in some cases can trigger the acute event itself. Recent studies have demonstrated that this phenomenon can be targeted diagnostically. Currently, existing point of care platforms for blood-based biomarker screening are largely geared towards immunoassay-based protein detection. Unfortunately, in part due to technological limitations, prior proteomic investigations in stroke have produced few candidate protein biomarkers with clinically useful levels of diagnostic accuracy.

### SUMMARY

Disclosed herein are methods that can comprise performing, using a computer processor, a random forest analysis on a first sample and a second sample. In some embodiments, a first sample and a second sample can be associated with an array. In some embodiments, a first sample can comprise a stroke patient biological sample. In some embodiments, a second sample can comprise a stroke mimic patient biological sample. In some embodiments, an array can comprise at least one protein probe. In some embodiments, a random forest analysis can comprise comparing a binding intensity level of antibodies in a first sample with at least one protein probe to a binding intensity level of antibodies in a second sample with at least one protein probe. In some embodiments, a random forest analysis can comprise generating a gini impurity score between a first sample and a second sample for at least one protein probe. In some embodiments, a method can further comprise performing multiple iterations of a random forest analysis. In some embodiments, multiple iterations can minimize a gini impurity score between a first sample and a second sample for at least one protein probe. In some embodiments, at least one protein probe can comprise a plurality of protein probes; which can generate a plurality of gini impurity scores between a first sample and a second sample for a plurality of protein probes. In some embodiments, a method can further comprise performing, using a computer processor, a recursive analysis. In some embodiments, a recursive analysis can comprise ranking a plurality of gini impurity scores. In some embodiments, a recursive analysis can comprise grouping a first set of a plurality of protein probes that can be based on minimization of gini impurity scores between a first sample and a second sample to generate a first profile. In some embodiments, a recursive analysis can comprise comparing a first profile to a second profile that can comprise a second set of a plurality of protein probes. In some embodiments, a second set of a plurality of protein probes may not be grouped based on minimization of gini impurity scores between a first sample and a second sample. In some embodiments, a stroke patient biological sample can comprise a hemorrhagic stroke patient biological sample. In some embodiments, a stroke patient biological sample can comprise an ischemic stroke patient biological sample. In some embodiments, an array can comprise at least 100,000 protein probes.

Also disclosed herein are systems for detecting stroke in a subject. In some embodiments, a system can comprise a memory that can store executable instructions. In some embodiments, a system can comprise a computer processor that can execute instructions to perform a method described herein. In some embodiments, a system can further comprise an integrated storage device.

Also disclosed herein are methods that can comprise contacting a sample with a synthetic protein. In some embodiments, a method can comprise detecting a binding intensity level of antibodies in a sample with a synthetic protein. In some embodiments, a method can comprise comparing a binding intensity level to a reference. In some embodiments, a reference can comprise a reference binding intensity level or a derivative thereof of antibodies in a stroke mimic sample with a synthetic protein. In some embodiments, a sample can be obtained from a subject. In some embodiments, a subject can have or may be suspected of having a stroke. In some embodiments, a synthetic protein can comprise an amino acid sequence at least 80% identical to any one of SEQ ID NO:1 to SEQ ID NO:50. In some embodiments, a synthetic protein can comprise an amino acid sequence at least 80% identical to SEQ ID NO: 1 or SEQ ID NO: 2. In some embodiments, a binding intensity level can be at least about 1.5 fold higher than a reference binding intensity level. In some embodiments, a binding intensity level can be at least about 1.5 fold lower than a reference binding intensity level. In some embodiments, a method can further comprise identifying a sample as a stroke sample or a stroke mimic sample. In some embodiments, an identifying can be with a sensitivity of at least 87% and a specificity of at least 87%. In some embodiments, a method can comprise identifying a sample as a stroke sample. In some embodiments, an identifying can be with a sensitivity of at least 90%. In some embodiments, an identifying can be with a specificity of at least 90%. In some embodiments, a method can comprise identifying a sample as a stroke mimic sample. In some embodiments, an identifying can be with a sensitivity of at least 90%. In some embodiments, an identifying can be with a specificity of at least 90%.

Also disclosed herein are methods that can comprise contacting a sample with one or more synthetic proteins. In some embodiments, one or more synthetic proteins can comprise an amino acid sequence at least 80% identical to any one of SEQ ID NO:1 to SEQ ID NO:50. In some embodiments, a method can comprise detecting a binding intensity level of antibodies in a sample with one or more synthetic proteins. In some embodiments, a sample can be obtained from a subject that can have a stroke or may be suspected of having a stroke. In some embodiments, one or more synthetic proteins can comprise an amino acid sequence at least 80% identical to any one of SEQ ID NO:1 to SEQ ID NO:17. In some embodiments, one or more synthetic proteins can comprise two or more amino acid sequence at least 80% identical to any one of SEQ ID NO:1 to SEQ ID NO:17. In some embodiments, one or more synthetic proteins can comprise three or more amino acid sequence at least 80% identical to any one of SEQ ID NO:1 to SEQ ID NO:17. In some embodiments, one or more synthetic proteins can comprise at least seventeen different synthetic proteins. In some embodiments, one or more synthetic proteins can comprise an amino acid sequence at least 80% identical to SEQ ID NO:1 or SEQ ID NO:2. In some embodiments, one or more synthetic proteins can comprise an amino acid sequence at least 95% identical to SEQ ID NO:1 or SEQ ID NO:2. In some embodiments, a method can further comprise comparing a binding intensity level to a reference. In some embodiments, a reference can comprise a reference binding intensity level or a derivative thereof of antibodies in an ischemic stroke sample, homographic stroke sample or stroke mimic sample with the one or more synthetic proteins. In some embodiments, a binding intensity level can be at least about 1.5 fold higher than a reference binding intensity level. In some embodiments, a binding intensity level can be at least about 1.5 fold lower than a reference binding intensity level. In some embodiments, a method can further comprise identifying a sample as an ischemic stroke sample, a hemorrhagic stroke sample or a stroke mimic sample. In some embodiments, an identifying can be with a sensitivity of at least 87% and a specificity of at least 87%. In some embodiments, a method can comprise identifying a sample as an ischemic stroke sample. In some embodiments, an identifying can be with a sensitivity of at least 90%. In some embodiments, an identifying can be with a specificity of at least 90%. In some embodiments, a method can comprise identifying a sample as a stroke mimic sample or a stroke sample. In some embodiments, an identifying can be with a sensitivity of at least 90%. In some embodiments, an identifying can be with a specificity of at least 90%. In some embodiments, a method can comprise identifying a sample as a hemorrhagic stroke sample. In some embodiments, an identifying can be with a sensitivity of at least 87%. In some embodiments, an identifying can be with a specificity of at least 87%.

Also disclosed herein are kits that can comprise a synthetic protein that can comprise an amino acid sequence at least 80% identical to any one of SEQ ID NO:1 to SEQ ID NO: 50. In some embodiments, a kit can comprise a detecting reagent for detecting binding of an antibody with a synthetic protein. In some embodiments, a synthetic protein can comprise an amino acid sequence at least 80% identical to any one of SEQ ID NO: 1 or SEQ ID NO: 2. In some embodiments, a synthetic protein can comprise an amino acid at least 90% identical to any one of SEQ ID NO: 1 to SEQ ID NO: 50. In some embodiments, a detecting regent can comprise a secondary antibody. In some embodiments, a secondary antibody can comprise a fluorophore.

Also disclosed herein are synthetic proteins that can comprise an amino acid sequence at least 80% identical to any one of SEQ ID NO: 1 to SEQ ID NO: 50. In some embodiments, a synthetic protein can comprise an amino acid sequence at least 95% identical to any one of SEQ ID NO:1 to SEQ ID NO:50. In some embodiments, a synthetic protein can comprise an amino acid sequence at least 95% identical to any one of SEQ ID NO:1 to SEQ ID NO:17. In some embodiments, a synthetic protein can comprise an amino acid sequence at least 95% identical to SEQ ID NO: 1 or SEQ ID NO: 2. In some embodiments, a synthetic protein can be in an array. Further described herein is a composition comprising an amino acid sequence at least 80% identical to any one of SEQ ID NO: 1 to SEQ ID NO: 50. In some embodiments, a composition described herein further comprises a sample. In some embodiment, a sample described herein is a sample obtained from a subject having a stroke, had a stroke, is suspected of having a stroke, or is suspected of having had a stroke.

Further disclosed herein are methods. In some embodiments, a method disclosed herein comprises contacting a sample with a synthetic protein. In some embodiments, the synthetic protein comprises an amino acid sequence at least 80% identical to any one of SEQ ID NO:1 to SEQ ID NO: 50. In some embodiments, the method further comprises detecting a binding intensity level of antibodies in the sample with the synthetic protein. In some embodiments, the method further comprises comparing the binding intensity level to a reference. In some cases, the sample comprises cell-free nucleic acids. In some embodiments, the sample was obtained from a subject. In some embodiments, the subject has, is suspected of having a stroke, or is suspected of having had a stroke. In some embodiments, the reference is a control. In some embodiments, a reference disclosed herein is a non-stroke reference. In some embodiments, the reference is a reference binding intensity.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts an exemplary overview of a protein array assay.

FIG. 2 depicts an exemplary R script that can be employed to evaluate a probe importance via a Random Forest analysis, where “normalized_data.csv” is a comma delimited text file in which the first column contains the true clinical diagnosis of each subject, and the remaining columns contain the log₂ transformed normalized antibody binding intensity levels associated with each probe of the protein array.

FIG. 3 depicts an exemplary overview of an assay that can be used in triage to evaluate a potential stroke patient.

FIG. 4 depicts an exemplary R script that can be used for a recursive feature selection, where “ranked_peptides.csv” is a comma delimited text file in which the first column contains the true clinical diagnosis of each subject, and the remaining columns contain the log₂ transformed normalized antibody binding intensity levels associated with the top 1000 ranked probes, ordered starting with the top ranked probe in column two.

FIG. 5 depicts an exemplary R script that can be used for a permutation analysis, where “normalized_data.csv” is a comma delimited text file in which the first column contains the true clinical diagnosis of each subject, and the remaining columns contain the log₂ transformed normalized antibody binding intensity levels associated with each probe of the protein array.

FIGS. 6A-6C depict selection of top ranked probes. FIG. 6A shows top ranked probes, ordered by mean decrease Gini coefficient, averaged across five independent random forest models. FIG. 6B shows combined ability of the antibody binding intensity levels of the top 50 ranked probes to discriminate between stroke patients and stroke mimics using random forest, compared to those of probes selected at random. FIG. 6C shows combined ability of the antibody binding intensity levels of the top 50 ranked probes to identify hemorrhagic stroke patients using random forest compared to those of probes selected at random.

FIGS. 7A-7C depict diagnostic ability of the top 17 probes. FIG. 7A shows ROC curve depicting the combined ability of the antibody binding intensity levels of the top 17 ranked probes to discriminate between stroke patients and stroke mimics using random forest. FIG. 7B shows combined ability of the antibody binding intensity levels of the top 17 ranked probes to identify hemorrhagic stroke patients using random forest when considering the total subject pool. FIG. 7C shows combined ability of the antibody binding intensity levels of the top 17 ranked probes to identify hemorrhagic stroke patients using random forest when only considering subjects classified as stroke. AUC, area under curve.

FIGS. 8A-8B depict differential antibody binding across the top 17 probes. FIG. 8A shows antibody binding intensity levels of the top 17 probes associated with samples from ischemic stroke patients, hemorrhagic stroke patients, and stroke mimics. Binding intensity levels were statistically compared using one-way ANOVA and p values were corrected for multiple comparisons using the Benjamini-Hochberg method. Probes were hierarchically clustered by similarity in binding intensity levels as assessed by Spearman's rho. FIG. 8B shows classification of each subject in the total patient pool according to the final random forest model's most representative decision tree. Each dot represents a single subject. Superscript labels on probes indicate importance ranking.

FIG. 9 depicts an exemplary computer implement workflow. Components from a peripheral blood sample from a subject that are indicative of stroke can bind to a protein probe as described herein. Binding can be detected using an assay. With the aid of a computer processor, a subject can be distinguished as a stroke vs. nonstroke subject, and as a hemorrhagic vs. ischemic subject.

### DETAILED DESCRIPTION

### Overview

Provided herein are computer implemented methods and systems for identifying probes for detection of biomarkers implicated in stroke. A method provided herein can be performed using a computer processor to perform a random forest analysis. A random forest analysis can be performed on one or more samples for example, a stroke patient biological sample or a stroke mimic patient biological sample. A binding intensity level of a component of a sample with at least one probe can be determined. Further, a binding intensity level can be compared between one or more samples to generate a gini impurity score between the one or more samples.

Also provided herein are systems for detecting stroke in a subject. A system can comprise executable instructions stored on computer readable memory to perform a method described herein. In some cases, the system can comprise a computer processor that can execute instructions to perform a method as described herein.

Also provided herein are methods that can comprise contacting one or more samples with a probe. A probe can be a synthetic protein having about 10-20 amino acids in length. With regard to the sample, the sample can be a biological sample comprising one or more antibodies. A method disclosed herein can include detecting a binding intensity level of an antibody with a probe. In some instances, a binding intensity level can be compared to a reference to determine if a sample is an ischemic stroke sample, a hemorrhagic stroke sample, a stroke mimic sample, or a non-stroke sample. The reference can include a binding intensity level or a derivative thereof of an antibody from a stroke mimic sample, a hemorrhagic stroke sample, an ischemic stroke sample, or a non-stroke sample control.

Also provided herein are kits for detecting stroke. A kit can include a probe as described herein that can binding to a component of a sample. A kit can include a detecting reagent that can detect binding of a component of a sample with a probe as described herein. In some cases, a probe can be used as a companion diagnostic. A kit can include instructions for administering a therapeutic based on the binding of a probe with a component of a sample.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal or a non-mammal. A mammal can be any member of the Mammalian class, including but not limited to a human; a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish, an insect, and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having or is having, a condition or disease. In some instances a disease or condition can be disruption of a BBB, a hemorrhagic stroke, or an ischemic stroke. A subject can be a patient. A subject can be an individual. In some instances, a subject, patient or individual can be used interchangeably.

The term “stroke” can refer to a condition of poor blood flow in a brain in a subject. In some cases, a stroke can result in cell death in a subject. In some cases, a stroke can be an ischemic stroke. An ischemic stroke can be a condition in which a decrease or loss of blood in an area of a brain that can result in tissue damage or destruction. In some cases, a stroke can be a hemorrhagic stroke. A hemorrhagic stroke can be a condition in which bleeding in a brain or an area around a brain can result in tissue damage or destruction. In some cases, a stroke can result in a reperfusion injury. A reperfusion injury can include inflammation, oxidative damage, hemorrhagic transformation, and the like. In some cases, a stroke can result in a disruption of a blood-brain barrier. In some cases, a stroke may not result in a disruption of a blood-brain barrier. The term “stroke mimic” can refer to a subject displaying a stroke-mimicking symptom who has not suffered a stroke. Stroke-mimicking symptoms can include pain, headache, aphasia, apraxia, agnosia, amnesia, stupor, confusion, vertigo, coma, delirium, dementia, seizure, migraine insomnia, hypersomnia, sleep apnea, tremor, dyskinesia, paralysis, visual disturbances, diplopia, paresthesias, dysarthria, hemiplegia, hemianesthesia, and hemianopia.

The terms “biomarker” and “biomarkers” can be used interchangeably to refer to one or more biomolecules. In some cases, a biomarker can be a biomolecule associated with a disease. When associated with a disease, a biomarker can have a profile different under the disease condition compared to a non-disease condition. Biomarkers can be any class of biomolecules, including polynucleotides, proteins, carbohydrates and lipids. In some cases, a biomarker can be a protein. A polypeptide or protein can be contemplated to include any fragments thereof, in particular, immunologically detectable fragments. A biomarker can also include one or more fragments of the biomarker having sufficient sequence such that it still possesses the same or substantially the same function as the full-size biomarker. An active fragment of a biomarker retains 100% of the activity of the full-size biomarker, or at least about 99%, 95%, 90%, 85%, 80% 75%, 70%, 65%, 60%, 55%, or at least 50% of its activity. In certain cases, an active fragment of a biomarker can be detectable (e.g., a protein detectable by an antibody, or a polynucleotide detectable by a labeled or unlabeled oligonucleotide).

### Methods

Provided herein are methods of assessing stroke in a subject (e.g., a subject suspected of having a stroke, a subject suspected of having had a stroke, a subject previously diagnosed with a stroke, etc). A method can comprise assessing stroke by contacting a sample with one or more probes.

The methods disclosed herein can be used to distinguish a stroke. In some cases, a biomarker present in a biological sample can be used to distinguish a subject displaying a stroke from a subject not displaying a stroke (e.g. a stroke mimic). In some cases, a biomarker for a stroke can be used to distinguish a subject displaying an ischemic stroke from a subject displaying a hemorrhagic stroke. In some cases, a biomarker can be used to distinguish a subjects displaying ischemic stroke, hemorrhagic stroke, and stroke mimics from each other.

In some cases, a biomarker can be present in a biological sample obtained or derived from a subject. A biological sample may be blood or any excretory liquid. Non-limiting examples of the biological sample may include saliva, blood, serum, cerebrospinal fluid, semen, feces, plasma, urine, a suspension of cells, or a suspension of cells and viruses. A biological sample may contain whole cells, lysed cells, plasma, red blood cells, platelets, skin cells, proteins, nucleic acids (e.g. DNA, RNA, maternal DNA, maternal RNA), circulating nucleic acids (e.g. cell-free nucleic acids, cell-free DNA/cfDNA, cell-free RNA/cfRNA), circulating tumor DNA/ctDNA, cell-free fetal DNA/cffDNA). As used herein, the term “cell-free” refers to the condition of the nucleic acid sequence as it appeared in the body before the sample is obtained from the body. For example, circulating cell-free nucleic acid sequences in a sample may have originated as cell-free nucleic acids circulating in the bloodstream of the human body. In contrast, nucleic acids that are extracted from a solid tissue, such as a biopsy, are generally not considered to be “cell-free.” In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid may be isolated from a blood sample. A cell-free nucleic acid may be isolated from a plasma sample. A cell-free nucleic acid may comprise a complementary DNA (cDNA). In some cases, one or more cDNAs may form a cDNA library.

In some instances, a sample can contain peptides or proteins. As used herein, the term “protein” can include any chain of two or more amino acids and can include peptides. In some instances where a biological sample is a blood sample, a protein can be a circulating protein. As used herein, a “circulating protein” can refer to proteins such as blood, plasma or serum proteins that are present in blood plasma. Examples of classes of blood proteins can include albumins, globulins, fibrinogen, lipoproteins, regulatory proteins, clotting factors, and the like. In some cases, a circulating protein can include a prealbumin such as transthyretin; alpha 1 antitrypsin; alpha 1 acid glycoprotein; alpha 1 fetoprotein; alpha2-macroglobulin; a gamma globulin; beta-2 microglobulin; haptoglobin; ceruloplasmin; complement component 3; complement component 4; C-reactive protein (CRP); a lipoprotein such as a chylomicrons, VLDL, LDL, or HDL; transferrin; prothrombin; MBL; MBP; and the like.

In some cases a circulating protein can be a globulin. A globulin can include an alpha 1 globulin such as alpha 1-antitrypsin, alpha 1-antichymotrypsin, orosomucoid (acid glycoprotein), serum amyloid A, or alpha 1-lipoprotein; an alpha 2 globulin such as haptoglobin, alpha-2u globulin, alpha 2-macroglobulin, ceruloplasmin, thyroxine-binding globulin, alpha 2-antiplasmin, protein C, alpha 2-lipoprotein, or angiotensinogen; a beta globulin such as beta-2 microglobulin, plasminogen, an angiostatin, properdin, a sex hormone-binding globulin, or transferrin; or a gamma globulin such as an immunoglobulin.

In some cases, a sample can comprise immunoglobulins. As used herein, the terms “immunoglobulin” and “antibody” can be used interchangeably to describe a protein used by the immune system to neutralize a pathogen or perceived pathogen. In some cases, a sample containing an antibody signature can be indicative of a disease state. For example, a sample can contain an antibody signature that can be indicative of an ischemic stroke, a hemorrhagic stroke, a stroke mimic, or any combination thereof.

Methods disclosed herein can assess stroke with high specificity and sensitivity. In some cases, a method can comprise one or more steps of: (a) contacting a sample with a probe, (b) detecting a binding intensity level of an antibody in the sample with a probe, and (c) comparing the binding intensity level to a reference. Such a method can be used to distinguish between a subject with an ischemic stroke, a subject with a hemorrhagic stroke, and a subject who is a stroke mimic, in a rapid fashion (e.g. in a triage). In some embodiments, a presence or absence of a stroke disruption can be determined based on a presence or level of an antibody in the sample.

A probe that can be employed in a method described herein can include a molecule that can bind to a component of a sample as described herein. A probe can be a macromolecule such as a nucleic acid or protein that can bind a component of a sample. A nucleic acid probe can include a nucleic-acid fragment that is at least partially complementary to another nucleic-acid sequence in a sample. In some instances, a nucleic acid probe can be labeled (e.g. fluorescent or radio label) in order to detect a binding of the probe with a component of a sample. A nucleic acid probe can be a fragment of DNA or RNA of variable length. In some cases, a nucleic acid probe can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length.

In some cases, a probe can be a protein. The terms “peptide,” “peptide,” and “polypeptide” can be used interchangeably to encompass both naturally-occurring and non-naturally occurring or synthetic proteins, and fragments, mutants, derivatives and analogs thereof. A protein may be monomeric or polymeric. Further, a protein may comprise a number of different domains each of which has one or more distinct activities. For the avoidance of doubt, a “protein” may be any length greater two amino acids. A protein can comprise an overall charge based on pKa of side chains of component amino acids. In some instances, a protein can have an overall positive charge. In some instances, a protein can have an overall negative charge. In some instances, a protein can have an overall neutral charge. A protein can furthermore exist as a zwitterion.

In some embodiments a probe can be a synthetic protein. A synthetic protein can be of variable length. In some cases, a synthetic protein can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 amino acids in length. In some cases, a synthetic protein can be from about 5 to about 50, from about 5 to about 45, from about 5 to about 40, from about 5 to about 35, from about 5 to about 30, from about 5 to about 25, from about 5 to about 20, from about 5 to about 15, or from about 5 to about 10 amino acids in length. In some cases, a synthetic protein can be from about 3 to about 25, from about 4 to about 25, from about 5 to about 25, from about 6 to about 25, from about 7 to about 25, from about 8 to about 25, from about 9 to about 25, from about 10 to about 25, from about 11 to about 25, from about 12 to about 25, from about 13 to about 25, from about 14 to about 25, from about 15 to about 25, from about 16 to about 25, from about 17 to about 25, from about 18 to about 25, from about 19 to about 25, from about 20 to about 25, from about 21 to about 25, from about 22 to about 25, from about 23 to about 25, or from about 24 to about 25 amino acids in length. In some cases, a protein can be no more than about 15 amino acids in length.

In some cases, at least one protein can be used to distinguish between disease states. In some cases, a method can employ at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 proteins. In some cases, a method can employ at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 proteins. In some cases, a method can employ at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, or 100000 proteins. In some cases, a method can employ at least about 105000, 110000, 115000, 120000, 125000, 130000, 135000, 140000, 145000, 150000, 155000, 160000, 165000, 170000, 175000, 180000, 185000, 190000, 195000, or 200000 proteins. In some cases, a method can employ from about 5 to about 50, from about 5 to about 45, from about 5 to about 40, from about 5 to about 35, from about 5 to about 30, from about 5 to about 25, from about 5 to about 20, from about 5 to about 15, or from about 5 to about 10 proteins. In some cases, a method can employ from about 1 to about 20, from about 1 to about 19, from about 1 to about 18, from about 1 to about 17, from about 1 to about 16, from about 1 to about 15, from about 1 to about 14, from about 1 to about 13, from about 1 to about 12, from about 1 to about 11, from about 1 to about 10, from about 1 to about 9, from about 1 to about 8, from about 1 to about 7, from about 1 to about 6, from about 1 to about 5, from about 1 to about 4, from about 1 to about 3, or from about 1 to about 2 proteins. In some cases, one or more proteins can be present on an array.

Exemplary proteins can include any of the proteins recited in Table 1 below.

TABLE 1

| Identifier | Sequence |
|---|---|
| SEQ ID NO: 1 | NVAVAQDENLAG |
| SEQ ID NO: 2 | NNYWANVASGLG |
| SEQ ID NO: 3 | QSLKPKGVALSG |
| SEQ ID NO: 4 | GASVHDGVALSG |
| SEQ ID NO: 5 | GEYFRWNWDSVA |
| SEQ ID NO: 6 | APFGQKDVALGL |
| SEQ ID NO: 7 | GDRRPLGVALSG |
| SEQ ID NO: 8 | KGQRGYHLKHDA |
| SEQ ID NO: 9 | AEQREFNKHLSA |
| SEQ ID NO: 10 | PEFRELSKHDVA |
| SEQ ID NO: 11 | PKPHGFPGQEYV |
| SEQ ID NO: 12 | KPEKLNGVALSG |
| SEQ ID NO: 13 | NSLKENGVALSG |
| SEQ ID NO: 14 | VLGPRHEPDSGA |
| SEQ ID NO: 15 | EKLYYHDSQEKH |
| SEQ ID NO: 16 | AWQKSKGVALSG |
| SEQ ID NO: 17 | QRPDPKDGQAKD |
| SEQ ID NO: 18 | DNEHWVLSGVAL |
| SEQ ID NO: 19 | HQVGRDPAGLSG |
| SEQ ID NO: 20 | KHPDYGQFRNLG |
| SEQ ID NO: 21 | VAPQQFEPKDVA |
| SEQ ID NO: 22 | RGGENEASVALG |
| SEQ ID NO: 23 | GRNPALQWNKDV |
| SEQ ID NO: 24 | QYDQHRYVAPHG |
| SEQ ID NO: 25 | NGRGVGQREGVG |
| SEQ ID NO: 26 | NHWYFVAGVLSG |
| SEQ ID NO: 27 | KWEKRKGVALSG |
| SEQ ID NO: 28 | EGNNLGEHALSS |
| SEQ ID NO: 29 | NYDDNHLGALSG |
| SEQ ID NO: 30 | GNDWFYHVALSG |
| SEQ ID NO: 31 | KNLWAHDLSGLG |
| SEQ ID NO: 32 | KVKNENALSASG |
| SEQ ID NO: 33 | RNGKRKVRWSVS |
| SEQ ID NO: 34 | EGGRFKSGVALS |
| SEQ ID NO: 35 | PPWARDVNVSAL |
| SEQ ID NO: 36 | PDADNDEKGVSG |
| SEQ ID NO: 37 | FSGLWAGEPWVG |
| SEQ ID NO: 38 | FHKQLQNKHLVG |
| SEQ ID NO: 39 | DHRELNALVALS |
| SEQ ID NO: 40 | HSHYHQRPYSLS |
| SEQ ID NO: 41 | YHFEKEKENHVA |
| SEQ ID NO: 42 | QHSKQPWNGKVS |
| SEQ ID NO: 43 | WGFVDAQWDGAG |
| SEQ ID NO: 44 | RQLAALGVALSG |
| SEQ ID NO: 45 | PEQRDQYLGASG |
| SEQ ID NO: 46 | WFHDADFRSVLS |
| SEQ ID NO: 47 | KYWFYAFPNKGS |
| SEQ ID NO: 48 | DHAGVYHSVALS |
| SEQ ID NO: 49 | PEFKQHSGVASG |
| SEQ ID NO: 50 | LGEDLSAFEKGV |

In some cases, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 proteins from Table 1 can be employed in a method described herein.

In some cases, a protein can comprise an amino acid sequence of any one of SEQ ID NO:1 to SEQ ID NO:50. In some cases, a protein can have an amino acid sequence with homology or sequence identity to any one of SEQ ID NO:1 to SEQ ID NO:50. The term “homology” can refer to a % sequence similarity of a protein to a reference protein. Homology can be calculated, for example, using a Smith-Waterman homology calculator. The term “sequence identity” can refer to % identity of a protein to a reference protein. In some cases, a protein can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology or sequence identity to any one of SEQ ID NO:1 to SEQ ID NO:50. In some cases, a protein can have from about 60% to about 100%, from about 65% to about 100%, from about 70% to about 100%, from about 75% to about 100%, from about 80% to about 100%, from about 85% to about 100%, from about 90% to about 100%, or from about 95% to about 100% homology or sequence identity to any one of SEQ ID NO:1 to SEQ ID NO:50.

The term “sequence identity,” as used herein, may be to calculations of “sequence identity” or “percent identity” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % sequence identity=(#of identical positions/total #of positions)×100). For example, a position in the first sequence may be occupied by the same nucleotide as the corresponding position in the second sequence, then the molecules are identical at that position. The percent sequence identity between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine sequence identity between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

A method described herein can include determining a binding intensity level of a component of a sample (e.g. an antibody) with a protein probe as described herein. Any conventional protein detection method can be used to measure a binding intensity level. Methods can include analytic biochemical methods such as electrophoresis, capillary electrophoresis, high performance liquid chromatography (HPLC), thin layer chromatography (TLC), hyperdiffusion chromatography, mass spectroscopy, spectrophotometry, electrophoresis (e.g., gel electrophoresis), and the like. Direct binding can be measured using techniques such as an immunoassay. Examples of immunoassays include immunoprecipitation, particle immunoassays, immunonephelometry, radioimmunoassays, enzyme immunoassays (e.g., ELISA), fluorescent immunoassays, chemiluminescent immunoassays, and Western blot analysis.

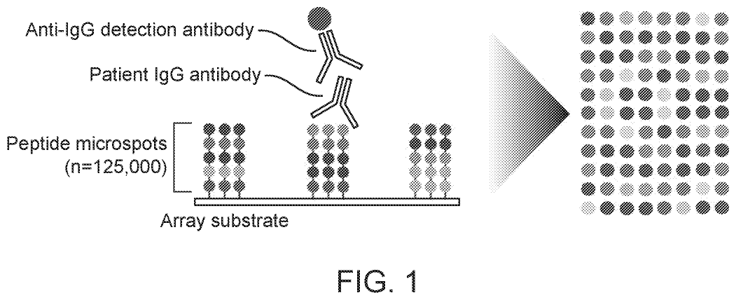

In one exemplary method, an array can be exposed to serum or soluble whole blood fractions to allow for protein-antibody interaction, rinsed, and bound antibodies can be subsequently detected with fluorescently-labeled pan anti-IgG antibody. FIG. 1 is illustrative of a concept in which an exemplary pattern of binding across the array can then be analyzed, giving a high-resolution profile of the composition of the circulating antibody pool.

In some embodiments, after determining a binding intensity level of components in a sample with a protein probe, the level can be compared to a reference. A reference can be a binding intensity level of components from reference sample (e.g. obtained from any reference subject), e.g., a healthy subject, an ischemic stroke subject, a hemorrhagic stroke subject, and/or a stroke mimic subject.

A binding intensity level can be determined in a triage setting to assess a stroke or a stroke mimic. In some cases, a binding intensity level can be determined once prior to a treatment. In some cases, a binding intensity level can be determined multiple times. In some cases, a binding intensity level can be determined multiple times over a period of time to monitor a progression of a disease state. In some cases, a binding intensity level can be determined at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 times within a time period of at least of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 hours.

In some cases, an antibody signature can be present in a subject displaying an ischemic stroke compared to a subject that does not display an ischemic stroke. In some cases, an antibody signature can be present in a subject displaying a hemorrhagic stroke compared to a subject that does not display a hemorrhagic stroke. In some cases, an antibody signature can be present in a subject that is a stroke mimic compared to a subject that is not a stroke mimic.

In some exemplary embodiments, an antibody derived from an ischemic stroke sample that binds to a probe can be present in a hemorrhagic stroke sample. In some exemplary embodiments, an antibody derived from an ischemic stroke sample that binds to a probe may be absent in a hemorrhagic stroke sample. In some exemplary embodiments, an antibody derived from an ischemic stroke sample that binds to a probe can be present in a stroke mimic sample. In some exemplary embodiments, an antibody derived from an ischemic stroke sample that binds to a probe may be absent in a stroke mimic sample. In some exemplary embodiments, an antibody derived from a hemorrhagic stroke sample that binds to a probe can be present in an ischemic stroke sample. In some exemplary embodiments, an antibody derived from a hemorrhagic stroke sample that binds to a probe can be absent in an ischemic stroke sample. In some exemplary embodiments, an antibody derived from a stroke mimic sample that binds to a probe can be present in an ischemic stroke sample. In some exemplary embodiments, an antibody derived from a stroke mimic sample that binds to a probe can be absent in an ischemic stroke sample. In some exemplary embodiments, an antibody derived from a stroke mimic sample that binds to a probe can be present in a hemorrhagic stroke sample. In some exemplary embodiments, an antibody derived from a stroke mimic sample that binds to a probe can be absent in a hemorrhagic stroke sample. In some exemplary embodiments, an antibody derived from a hemorrhagic stroke sample that binds to a probe and is present in an ischemic stroke sample can be present in a stroke mimic sample. In some exemplary embodiments, an antibody derived from a hemorrhagic stroke sample that binds to a probe and is present in an ischemic stroke sample can be absent in a stroke mimic sample. In some exemplary embodiments, an antibody derived from a stroke mimic sample that binds to a probe can be present in an ischemic stroke sample and a hemorrhagic stroke sample. In some exemplary embodiments, an antibody derived from a stroke mimic sample that binds to a probe may be absent in an ischemic stroke sample and a hemorrhagic stroke sample.

A sample can be fresh or frozen, and/or can be treated, e.g. with heparin, citrate, or EDTA. A sample can also include sections of tissues such as frozen sections taken for histological purposes. A sample can be obtained from a subject prior to the subject exhibiting a stroke or a symptom of stroke. In some cases, a sample can be obtained from a subject prior to or after the subject exhibiting a hemorrhagic transformation. In some cases, a sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours from the onset of a symptom or a hemorrhagic transformation. A sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours from the onset of a symptom of a stroke. A sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours prior to the onset of a symptom of a stroke or hemorrhagic transformation.

In some cases, a sample can be a biological fluid. When a sample is a biological fluid, the volume of the fluidic sample can be greater than 1 mL (milliliter). In some cases, the volume of the fluidic sample can be within a range of at least about 1.0 mL to at least about 15 mL. For example, the volume of the sample can be about 1.0 mL, 1.1 mL, 1.2 mL, 1.4 mL, 1.6 mL, 1.8 mL, 1.9 mL, 2 mL, 3 mL, 4 mL, 5 mL, 6 mL, 7 mL, 8 mL, 9 mL, or 10 mL. Alternatively, in some cases, the volume of the fluidic sample can be no greater than 1 mL. For example, the volume of the sample can be less than about 0.00001 mL, 0.0001 mL, 0.001 mL, 0.01 mL, 0.1 mL, 0.2 mL, 0.4 mL, 0.6 mL, 0.8 mL, or 1 mL.

A sample disclosed herein can be blood. For example, a sample can be peripheral blood. In some cases, a sample can be a fraction of blood. In one example, a sample can be serum. In another example, a sample can be plasma. In another example, a sample can include one or more cells circulating in blood. Such cells can include red blood cells (e.g., erythrocytes), white blood cells (e.g., leukocytes, including, neutrophils, eosinophils, basophils, lymphocyte, and monocytes (e.g., peripheral blood mononuclear cell)), platelets (e.g., thrombocytes), circulating tumor cells, or any type of cells circulating in peripheral blood and combinations thereof.

A sample can be derived from a subject. In some cases, a subject can be a human, e.g. a human patient. In some cases, a subject can be a non-human animal, including a mammal such as a domestic pet (e.g., a dog, or a cat) or a primate. A sample can contain one or more polypeptide or protein biomarkers, or a polynucleotide biomarker disclosed herein (e.g., mRNA). A subject can be suspected of having a condition (e.g., a disease).

Stroke can refer to a medical condition that can occur when the blood supply to part of the brain may be interrupted or severely reduced, depriving brain tissue of oxygen and nutrients. Within minutes, brain cells can begin to die. Stroke can include ischemic stroke, hemorrhagic stroke and transient ischemic attack (TIA). Ischemic stroke can occur when there can be a decrease or loss of blood flow to an area of the brain resulting in tissue damage or destruction. Hemorrhagic stroke can occur when a blood vessel located in the brain is ruptured leading to the leakage and accumulation of blood directly in the brain tissue. Transient ischemic attack or mini stroke, can occur when a blood vessel is temporarily blocked. Ischemic stroke can include thrombotic, embolic, lacunar and hypoperfusion types of strokes.

An ischemic stroke subject can refer to a subject with an ischemic stroke or having a risk of having an ischemic stroke. In some cases, an ischemic stroke subject can be a subject that has had ischemic stroke within 24 hours. In a particular example, an ischemic stroke subject can be a subject that has had an ischemic stroke within 4.5 hours. A non-ischemic stroke subject can be a subject who has not had an ischemic stroke. In some cases, a non-ischemic stroke subject can be a subject who has not had an ischemic stroke and has no risk of having an ischemic stroke.

A subject with stroke (e.g., ischemic stroke) can have one or more stroke symptoms. Stroke symptoms can be present at the onset of any type of stroke (e.g., ischemic stroke or hemorrhagic stroke). Stroke symptoms can be present before or after the onset of any type of stroke. Stroke symptoms can include those symptoms recognized by the National Stroke Association, which include: (a) sudden numbness or weakness of the face, arm or leg—especially on one side of the body; (b) sudden confusion, trouble speaking or understanding; (c) sudden trouble seeing in one or both eyes; (d) sudden trouble walking, dizziness, loss of balance or coordination, and (e) sudden severe headache with no known cause.

A non-ischemic stroke subject can have stroke-mimicking symptoms. Stroke-mimicking symptoms can include pain, headache, aphasia, apraxia, agnosia, amnesia, stupor, confusion, vertigo, coma, delirium, dementia, seizure, migraine insomnia, hypersomnia, sleep apnea, tremor, dyskinesia, paralysis, visual disturbances, diplopia, paresthesia, dysarthria, hemiplegia, hemianesthesia, and hemianopia. When a stroke-mimicking symptom is present in a subject that has not suffered a stroke, the symptoms can be referred to as “stroke mimics”. Conditions within the differential diagnosis of stroke include brain tumor (e.g., primary and metastatic disease), aneurysm, electrocution, burns, infections (e.g., meningitis), cerebral hypoxia, head injury (e.g. concussion), traumatic brain injury, stress, dehydration, nerve palsy (e.g., cranial or peripheral), hypoglycemia, migraine, multiple sclerosis, peripheral vascular disease, peripheral neuropathy, seizure (e.g., grand mal seizure), subdural hematoma, syncope, and transient unilateral weakness. Biomarkers (e.g. antibodies) of ischemic stroke can be those that can distinguish acute ischemic stroke from these stroke-mimicking conditions and/or from hemorrhagic stroke. In some cases, the biomarkers can identify a stroke mimicking condition disclosed herein. In some cases, the biomarkers can identify a non-stroke condition disclosed herein.

The methods, systems and kits disclosed herein can be used to assess a condition. A condition can be a disease or a risk of a disease in a subject. For example, the methods can determining a presence group of biomarkers using a probe as described herein in a sample from a subject, and assessing a disease or a risk of a disease in a subject based on the expression. In some cases, a condition can be a risk factor for stroke, e.g., high blood pressure, atrial fibrillation, high cholesterol, diabetes, atherosclerosis, circulation problems, tobacco use, alcohol use, physical inactivity, obesity, age, gender, race, family history, previous stroke, previous transient ischemic attack (TIA), fibromuscular dysplasia, patent foramen ovale, or any combination thereof. If one or more risk factors are known in a subject, the risk factors can be used, e.g., in combination with methods described herein, to assess a risk of ischemic stroke or hemorrhagic stroke in the subject.

A condition can be a disease. A disease can be stroke or stroke associated disease. A disease can be ischemic stroke. In some cases, a disease can be Alzheimer's disease or Parkinson's disease. In some cases, a disease can be an autoimmune disease such as acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, conginital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lyme disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease can be a cancer such as Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain tumor, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, childhood, Carcinoid tumor, gastrointestinal, Carcinoma of unknown primary, Central nervous system lymphoma, primary, Cerebellar astrocytoma, childhood, Cerebral astrocytoma/Malignant glioma, childhood, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) cancer, Gastrointestinal Carcinoid Tumor, Gastrointestinal stromal tumor (GIST), Germ cell tumor: extracranial, extragonadal, or ovarian, Gestational trophoblastic tumor, Glioma of the brain stem, Glioma, Childhood Cerebral Astrocytoma, Glioma, Childhood Visual Pathway and Hypothalamic, Gastric carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, childhood, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Marcus Whittle, Deadly Disease, Macroglobulinemia, Waldenström, Malignant Fibrous Histiocytoma of Bone/Osteosarcoma, Medulloblastoma, Childhood, Melanoma, Melanoma, Intraocular (Eye), Merkel Cell Carcinoma, Mesothelioma, Adult Malignant, Mesothelioma, Childhood, Metastatic Squamous Neck Cancer with Occult Primary, Mouth Cancer, Multiple Endocrine Neoplasia Syndrome, Childhood, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Chronic, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, childhood, Salivary gland cancer, Sarcoma, Ewing family of tumors, Sarcoma, Kaposi, Sarcoma, soft tissue, Sarcoma, uterine, Sézary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma—see Skin cancer (nonmelanoma), Squamous neck cancer with occult primary, metastatic, Stomach cancer, Supratentorial primitive neuroectodermal tumor, childhood, T-Cell lymphoma, cutaneous—see Mycosis Fungoides and Sézary syndrome, Testicular cancer, Throat cancer, Thymoma, childhood, Thymoma and Thymic carcinoma, Thyroid cancer, Thyroid cancer, childhood, Transitional cell cancer of the renal pelvis and ureter, Trophoblastic tumor, gestational, Unknown primary site, carcinoma of, adult, Unknown primary site, cancer of, childhood, Ureter and renal pelvis, transitional cell cancer, Urethral cancer, Uterine cancer, endometrial, Uterine sarcoma, Vaginal cancer, Visual pathway and hypothalamic glioma, childhood, Vulvar cancer, Waldenström macroglobulinemia, Wilms tumor (kidney cancer), childhood.

In some cases, a disease can be inflammatory disease, infectious disease, cardiovascular disease and metabolic disease. Specific infectious diseases include, but is not limited to AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, lyme disease, malaria, measles. Meningococcal disease, mumps, pertussis (whooping cough), plague, paralytic poliomyelitis, psittacosis, Q fever, rabies, rocky mountain spotted fever, rubella, conginital rubella syndrome (SARS), shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate resistant staphylocossus aureus, varicella, yellow fever, variant Creutzfeldt-Jakob disease (vCJD), Ebola hemorrhagic fever, Echinococcosis, Hendra virus infection, human monkeypox, influenza A, H5N1, lassa fever, Margurg hemorrhagic fever, Nipah virus, O'nyong fever, Rift valley fever, Venezuelan equine encephalitis and West Nile virus.

In some embodiments, the methods, device and kits described herein can detect one or more of the diseases disclosed herein. In some embodiments, one or more of the biomarkers disclosed herein can be used to assess one or more disease disclosed herein. In some embodiments, one or more of the biomarkers disclosed herein can be used to detect one or more diseases disclosed herein.

The presence or level of a biomarker can be measured using any suitable immunoassay, for example, enzyme-linked immunoassays (ELISA), radioimmunoassays (RIAs), competitive binding assays, and the like. Specific immunological binding of an antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The analysis of a plurality of biomarkers can be carried out separately or simultaneously with one test sample. For separate or sequential assay of biomarkers, suitable apparatuses can include clinical laboratory analyzers such as the ELECSYS® (Roche), the AXSYM® (Abbott), the ACCESS® (Beckman), the ADVIA® CENTAUR® (Bayer) immunoassay systems, the NICHOLS ADVANTAGE® (Nichols Institute) immunoassay system, etc. Apparatuses or protein chips or gene chips can perform simultaneous assays of a plurality of biomarkers on a single surface. Useful physical formats comprise surfaces having a plurality of discrete, addressable locations for the detection of a plurality of different analytes. Such formats can include protein microarrays, or “protein chips” (see, e.g., Ng and Ilag, J. Cell Mol. Med. 6: 329-340 (2002)) and certain capillary devices (see e.g., U.S. Pat. No. 6,019,944). In these embodiments each discrete surface location can comprise antibodies to immobilize one or more analyte(s) (e.g., a biomarker) for detection at each location. Surfaces can alternatively comprise one or more discrete particles (e.g., microparticles or nanoparticles) immobilized at discrete locations of a surface, where the microparticles comprise antibodies to immobilize one analyte (e.g., a biomarker) for detection. The protein biochips can further include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, each of which is incorporated by reference herein in its entirety.

The presence or level of a biomarker can be measured using any suitable immunoassay, for example, enzyme-linked immunoassays (ELISA), radioimmunoassays (RIAs), competitive binding assays, and the like. Specific immunological binding of an antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The analysis of a plurality of biomarkers can be carried out separately or simultaneously with one test sample. For separate or sequential assay of biomarkers, suitable apparatuses can include clinical laboratory analyzers such as the ELECSYS® (Roche), the AXSYM® (Abbott), the ACCESS® (Beckman), the ADVIA® CENTAUR® (Bayer) immunoassay systems, the NICHOLS ADVANTAGE® (Nichols Institute) immunoassay system, etc. Apparatuses or protein chips or gene chips can perform simultaneous assays of a plurality of biomarkers on a single surface. Useful physical formats comprise surfaces having a plurality of discrete, addressable locations for the detection of a plurality of different analytes. Such formats can include protein microarrays, or “protein chips” (see, e.g., Ng and Ilag, J. Cell Mol. Med. 6: 329-340 (2002)) and certain capillary devices (see e.g., U.S. Pat. No. 6,019,944). In these embodiments each discrete surface location can comprise proteins or antibodies to immobilize one or more analyte(s) (e.g., a biomarker) for detection at each location. Surfaces can alternatively comprise one or more discrete particles (e.g., microparticles or nanoparticles) immobilized at discrete locations of a surface, where the microparticles comprise antibodies to immobilize one analyte (e.g., a biomarker) for detection. The protein biochips can further include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, each of which is incorporated by reference herein in its entirety.

In some cases, probes that can bind to components of a sample indicative of a disease state can be identified. Probes can be identified using methods such as machine learning and/or pattern recognition. In some cases, probes can be identified based on a predictive model. Established statistical algorithms and methods useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (Log Reg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms can also be used in determining subject sub-groups.

In some instances, random forest analysis can be used for identification of probes. Random forests or random decision forests can be an ensemble learning method for classification, regression and other tasks, that operate by constructing a multitude of decision trees at training time and outputting the class that is the mode of the classes (classification) or mean prediction (regression) of the individual trees. Random decision forests can correct for decision trees' habit of overfitting to their training set.

A random forest analysis can include a decision tree or tree learning. A decision tree learning can use a decision tree (as a predictive model) to go from observations about an item to conclusions about the item's target value. In some cases, a decision trees can include a target variable that can take continuous values (typically real numbers). In some cases, a random forest analysis can include tree bragging.

As used herein, a random forest analysis can include comparing a binding intensity level of component in a first sample with a protein probe to a binding intensity level of components in a second sample with the protein probe to generate a gini impurity score between the first sample and the second sample for the protein probe. Gini impurity is a measure of how often a randomly chosen element from the set would be incorrectly labeled if it was randomly labeled according to the distribution of labels in the subset. Gini impurity can be computed by summing the probability of an item with label i being chosen times the probability of a mistake in categorizing that item. It reaches its minimum (zero) when all cases in the node fall into a single target category. A gini impurity score or gini coefficient can be used as a metric to determine the importance of a probe at distinguishing between a first and second sample (and thereby between a first and second disease state as described herein). In some cases, a decrease in gini score is used to determine the importance. In some embodiments, a probe can have a mean decrease in gini score of about 0.05, about 0.049, about 0.048, about 0.047, about 0.046, about 0.045, about 0.044, about 0.043, about 0.042, about 0.041, about 0.04, about 0.039, about 0.038, about 0.037, about 0.036, about 0.035, about 0.034, about 0.033, about 0.032, about 0.031, about 0.03, about 0.029, about 0.028, about 0.027, about 0.026, about 0.025, about 0.024, about 0.023, about 0.022, about 0.021, about 0.02, about 0.019, about 0.018, about 0.017, about 0.016, about 0.015, about 0.014, about 0.013, about 0.012, about 0.011, about 0.01, about 0.009, about 0.008, about 0.007, about 0.006, about 0.005, about 0.004, about 0.003, about 0.05, about 0.002, or about 0.001. In some embodiments, a probe can have a mean decrease in gini score of from about 0.005 to about 0.03, from about 0.006 to about 0.03, from about 0.007 to about 0.03, from about 0.008 to about 0.03, from about 0.009 to about 0.03, from about 0.01 to about 0.03, from about 0.011 to about 0.03, from about 0.012 to about 0.03, from about 0.013 to about 0.03, from about 0.014 to about 0.03, from about 0.015 to about 0.03, from about 0.016 to about 0.03, from about 0.017 to about 0.03, from about 0.018 to about 0.03, from about 0.019 to about 0.03, from about 0.02 to about 0.03, from about 0.021 to about 0.03, from about 0.022 to about 0.03, from about 0.023 to about 0.03, from about 0.024 to about 0.03, from about 0.025 to about 0.03, from about 0.026 to about 0.03, from about 0.027 to about 0.03, from about 0.028 to about 0.03, or from about 0.029 to about 0.03.

In some cases, multiple iterations of random forest can be employed in order to distinguish between disease states to achieve optimum parameters or metrics. Parameters to be measured include those described in Fischer et al., Intensive Care Med. 29: 1043-51, 2003, which is incorporated herein in its entirety. These parameters include sensitivity and specificity, predictive values, likelihood ratios, diagnostic odds ratios, and receiver operating characteristic (ROC) curve areas. One or a group of effective probes can exhibit one or more of the following results on these various parameters: at least 75% sensitivity, combined with at least 75% specificity; ROC curve area of at least 0.7, at least 0.8, at least 0.9, or at least 0.95; and/or a positive likelihood ratio (calculated as sensitivity/(1-specificity)) of at least 5, at least 10, or at least 20, and a negative likelihood ratio (calculated as (1-sensitivity)/specificity) of less than or equal to 0.3, less than or equal to 0.2, or less than or equal to 0.1. The ROC areas can be calculated and used in determining the effectiveness of a probe as described in US Patent Application Publication No. 2013/0189243, which is incorporated herein in its entirety.

Methods, systems and kits provided herein can distinguish between a condition such as ischemic stroke and hemorrhagic stroke in a subject, and can distinguish each from a stroke mimic subject with high specificity and sensitivity. As used herein, the term “specificity” can refer to a measure of the proportion of negatives that are correctly identified as such (e.g., the percentage of healthy people who are correctly identified as not having the condition). As used herein, the term “sensitivity” can refer to a measure of the proportion of positives that are correctly identified as such (e.g., the percentage of sick people who are correctly identified as having the condition). Methods, systems and kits provided herein can assess a condition (e.g., ischemic stroke, hemorrhagic stroke, or stroke mimic) in a subject with a specificity of at least about 75%, 80%, 85%, 90%, 95%, 96%, 97%, 98%, 99%, or 100%. Methods, devices and kits provided herein can assess a condition (e.g., ischemic stroke, hemorrhagic stroke, or stroke mimic) in a subject with a sensitivity of at least about 75%, 80%, 85%, 90%, 95%, 96%, 97%, 98%, 99%, or 100%. Methods, systems and kits provided herein can assess a condition (e.g., ischemic stroke, hemorrhagic stroke, or stroke mimic) in a subject with a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% a sensitivity of about 100%.

Methods described herein can be used to distinguish an ischemic stroke from a hemorrhagic stroke with high specificity and sensitivity. In some cases, the methods can distinguish an ischemic stroke from a hemorrhagic stroke in a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the use of no more than 1, 2, 3, 4, 5, 6, 7, 8, 9, 10, 11, 12, 13, 14, 15, 16, 17, 18, 19, or 20 protein probes. In some cases, the methods, systems and kits can distinguish an ischemic stroke from a hemorrhagic stroke with a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the use of at least 10 protein probes.

Methods of can be used to distinguish stroke (e.g. ischemic stroke or hemorrhagic stroke) from a stroke mimic as described herein with high specificity and sensitivity. In some cases, the methods can distinguish a stroke from a stroke mimic in a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the use of no more than 1, 2, 3, 4, 5, 6, 7, 8, 9, 10, 11, 12, 13, 14, 15, 16, 17, 18, 19, or 20 protein probes. In some cases, the methods, systems and kits can distinguish a stroke from a stroke mimic with a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the use of at least 10 protein probes.

A method can comprise administering a treatment to a subject deemed at risk of developing a stroke such as an ischemic stroke or a hemorrhagic stroke. Binding of a protein probe to a component of a subject sample may indicate that a subject will be responsive to a given treatment. In some cases the treatment is disclosed herein. In some cases, a subject pool (e.g. in a clinical trial) can be stratified into pools of subjects, some of which may be deemed to be responsive to treatment based on an assay as described herein. In some instances, stratification can be based on the binding of a protein probe to a component of a subject sample. In some cases, the methods can comprise administering a pharmaceutically effective dose of a drug or a salt thereof for treating ischemic stroke. In some embodiments, a drug for treating ischemic stroke can comprise a thrombolytic agent or antithrombotic agent. In some embodiments, a drug for treating ischemic stroke can be one or more compounds that are capable of dissolving blood clots such as psilocybin, tPA (Alteplase or Activase), reteplase (Retavase), tenecteplase (TNKasa), anistreplase (Eminase), streptoquinase (Kabikinase, Streptase) or uroquinase (Abokinase), and anticoagulant compounds, i.e., compounds that prevent coagulation and include, without limitation, vitamin K antagonists (warfarin, acenocumarol, fenprocoumon and fenidione), heparin and heparin derivatives such as low molecular weight heparins, factor Xa inhibitors such as synthetic pentasaccharides, direct thrombin inhibitors (argatroban, lepirudin, bivalirudin and ximelagatran) and antiplatelet compounds that act by inhibition of platelet aggregation and, therefore, thrombus formation and include, without limitation, cyclooxygenase inhibitors (aspirin), adenosine diphosphate receptor inhibitors (clopidrogrel and ticlopidine), phosphodiesterase inhibitors (cilostazol), glycoprotein inhibitors (Abciximab Eptifibatide, Tirofiban and Defibrotide) and adenosine uptake inhibitors (dipiridamol). The drug for treating ischemic stroke can be tissue plasminogen activator (tPA).

In some cases, a treatment can comprise endovascular therapy. In some cases, endovascular therapy can be performed after a treatment is administered. In some cases, endovascular therapy can be performed before a treatment is administered. In some cases, a treatment can comprise a thrombolytic agent. In some cases, an endovascular therapy can be a mechanical thrombectomy. In some cases, a stent retriever can be sent to the site of a blocked blood vessel in the brain to remove a clot. In some cases, after a stent retriever grasps a clot or a portion thereof, the stent retriever and the clot or portions thereof can be removed. In some cases, a catheter can be threaded through an artery up to a blocked artery in the brain. In some cases, a stent can open and grasp a clot or portions thereof, allowing for the removal of the stent with the trapped clot or portions thereof. In some cases, suction tubes can be used. In some cases, a stent can be self-expanding, balloon-expandable, and or drug eluting.

In some cases, the treatments disclosed herein may be administered by any route, including, without limitation, oral, intravenous, intramuscular, intra-arterial, intramedullary, intrathecal, intraventricular, transdermal, subcutaneous, intraperitoneal, intranasal, enteric, topical, sublingual or rectal route. A review of the different dosage forms of active ingredients and excipients to be used and their manufacturing processes is provided in “Tratado de Farmacia Galénica”, C. Fauli and Trillo, Luzán 5, S. A. de Ediciones, 1993 and in Remington's Pharmaceutical Sciences (A. R. Gennaro, Ed.), 20^{th} edition, Williams & Wilkins PA, USA (2000). Examples of pharmaceutically acceptable vehicles are known in prior art and include phosphate buffered saline solutions, water, emulsions, such as oil/water emulsions, different types of humectants, sterile solutions, etc. The compositions that comprise said vehicles may be formulated by conventional processes which are known in prior art.

In some cases, the methods can comprise administering a pharmaceutically effective dose of a drug for treating ischemic stroke within 24 hours, 12 hours, 11 hours, 10 hours, 9 hours, 8 hours, 7 hours, 6 hours, 5 hours, 4 hours, 3 hours, 2 hours, or 1 hour, 30 minutes, 20 minutes, or 10 minutes from the ischemic stroke onset. For example, the methods can comprise administering a pharmaceutically effective dose of a drug for treating ischemic stroke within 4.5 hours of ischemic stroke onset. In a particular example, the methods can comprise administering a pharmaceutically effective dose of tPA within 4.5 hours of ischemic stroke onset. In some cases, the methods can comprise determining whether or not to take the patient to neuro-interventional radiology for clot removal or intra-arterial tPA. In this particular example, the methods can comprise administering a pharmaceutically effective dose of intra-arterial tPA within 8 hours of ischemic stroke onset. In certain cases, the methods comprise administering a treatment to the subject if the level of the cell-free nucleic acids in the subject can be higher than a reference level. In some embodiments, a treatment may not be administered if the level of the cell-free nucleic acids in the subject is equal to or less than the reference. In some embodiments, a treatment can be administered if ischemic stroke, or BBB disruption is determined. In some cases, an identification of hemorrhagic transformation or BBB disruption can prevent the administration of a treatment, for example tPA.

### Kits

Provided herein are kits for detecting a stroke, for example, ischemic stroke or hemorrhagic stroke in a subject. A kit can be used for performing any methods described herein. For example, the kits can be used to determine an antibody signature indicative of a disease state in a subject. When assessing the condition with a kit, high specificity and sensitivity can be achieved. The kits can also be used to evaluate a treatment of a condition associated with stroke. For example, kits disclosed herein can comprise a panel of probes and a detecting reagent.

A kit can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 protein probes. In some cases, a kit can comprise at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 proteins. In some cases, a kit can comprise at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, or 100000 proteins. In some cases, a kit can comprise at least about 105000, 110000, 115000, 120000, 125000, 130000, 135000, 140000, 145000, 150000, 155000, 160000, 165000, 170000, 175000, 180000, 185000, 190000, 195000, or 200000 proteins. In some cases, a kit can comprise from about 5 to about 50, from about 5 to about 45, from about 5 to about 40, from about 5 to about 35, from about 5 to about 30, from about 5 to about 25, from about 5 to about 20, from about 5 to about 15, or from about 5 to about 10 proteins. In some cases, a kit can comprise from about 1 to about 20, from about 1 to about 19, from about 1 to about 18, from about 1 to about 17, from about 1 to about 16, from about 1 to about 15, from about 1 to about 14, from about 1 to about 13, from about 1 to about 12, from about 1 to about 11, from about 1 to about 10, from about 1 to about 9, from about 1 to about 8, from about 1 to about 7, from about 1 to about 6, from about 1 to about 5, from about 1 to about 4, from about 1 to about 3, or from about 1 to about 2 proteins. In some cases, the proteins described herein can be synthetic proteins.

A kit can comprise protein probes that can bind biomarkers such as antibodies that are indicative of a disease state. Such protein probes can include a protein recited in Table 1. In some cases, a kit can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 proteins from Table 1.

In some cases, a kit can comprise a protein with a protein sequence of any one of SEQ ID NO:1 to SEQ ID NO:50. In some cases, a kit can comprise a protein that can have an amino acid sequence with homology or sequence identity to any one of SEQ ID NO:1 to SEQ ID NO:50. Such a protein can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology or sequence identity to any one of SEQ ID NO:1 to SEQ ID NO:50. In some cases, such a protein can have from about 60% to about 100%, from about 65% to about 100%, from about 70% to about 100%, from about 75% to about 100%, from about 80% to about 100%, from about 85% to about 100%, from about 90% to about 100%, or from about 95% to about 100% homology or sequence identity to any one of SEQ ID NO:1 to SEQ ID NO:50.

The kits can comprise a probe that can bind (e.g., directly or indirectly) to at least one biomarker in the sample. The probes can be labeled. For example, the probes can comprise labels. The labels can be used to track the binding of the probes with biomarkers of blood brain barrier disruption in a sample. The labels can be fluorescent or luminescent tags, metals, dyes, radioactive isotopes, and the like. Examples of labels include paramagnetic ions, radioactive isotopes; fluorochromes, metals, dyes, NMR-detectable substances, and X-ray imaging compounds. Paramagnetic ions include chromium (III), manganese (II), iron (III), iron (II), cobalt (II), nickel (II), copper (II), neodymium (II), samarium (III), ytterbium (III), gadolinium (III), vanadium (II), terbium (III), dysprosium (III), holmium (III) and/or erbium (III). Ions useful in other contexts, such as X-ray imaging, include but are not limited to lanthanum (III), gold (III), lead (II), and especially bismuth (III). Radioactive isotopes include “-carbon, ¹⁵chromium, ³⁶-chlorine, ⁵⁷cobalt, and the like may be utilized. Among the fluorescent labels contemplated for use include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Enzymes (an enzyme tag) that will generate a colored product upon contact with a chromogenic substrate may also be used. Examples of suitable enzymes include urease, alkaline phosphatase, hydrogen peroxidase or glucose oxidase. Secondary binding ligands can be biotin and/or avidin and streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241.

The kits can further comprise a detecting reagent. The detecting reagent can be used for examining binding of the probes with the group of biomarkers. The detecting reagent can comprise any label described herein, e.g., a fluorescent or radioactive label. In some cases, the kits can also include an immunodetection reagent or label for the detection of specific immunoreaction between the provided biomarkers and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction can be detected or quantified by means of detecting or quantifying the label. Immunodetection reagents and processes suitable for application in connection with the novel methods disclosed herein are generally well known in the art.

The reagents can include ancillary agents such as buffering agents and protein stabilizing agents, e.g., polysaccharides and the like. The kit may further include where necessary agents for reducing background interference in a test, agents for increasing signal, apparatus for conducting a test, calibration curves and charts, standardization curves and charts, and the like.

The kits can further comprise a computer-readable medium for assessing a condition in a subject. For example, the computer-readable medium can analyze the difference between an antibody binding signature in a sample from a subject and a reference, thus assessing a condition in the subject. In some embodiments, a kit disclosed herein can comprise instructions for use.

### Systems for Distinguishing Stroke

Disclosed herein are systems for distinguishing a stroke subject (e.g. ischemic stroke or hemorrhagic stroke) from a stroke mimic subject. Such systems can comprise a memory that stores executable instructions. A memory can be computer readable. The systems can further comprise a processor that executes the executable instructions to perform the methods disclosed herein.

Disclosed herein are systems for detecting stroke, or a condition associated therewith, in a subject. The systems can comprise a memory that stores executive instruction and a processor that executes the executable instructions. The systems can be configured to perform any method of detecting stroke disclosed herein.

In some embodiments, a system can be configured to communicate with a database. In some embodiments, a system can transmit data to a database or server. A database or server can be a cloud server or database. In some embodiments, a system can transmit data wirelessly via a Wi-Fi, or Bluetooth connection. Databases can include functional or bioinformatics databases such as the Database for Annotation, Visualization and Integrated Discovery (DAVID); BioGraph, Entrez, GeneCards, Genome Aggregation Database, mGEN, MOPED, SOURCE, Rfam, DASHR, UnitProt, Pfam, Swiss-Prot Protein Knowledgebase, Protein Data Bank (PDB), and Structural Classification of Proteins (SCOP).

In some aspects, a system described herein can comprise centralized data processing, that could be cloud-based, internet-based, locally accessible network (LAN)-based, or a dedicated reading center using pre-existent or new platforms.

Binding of biomarkers such as antibodies from a sample to exemplary protein probes as described herein can be detected as described herein. The assay output can be fed into a system that can distinguish the biomarker binding profile from that of a control or reference. A result can be stored via local or cloud based storage for future use, and/or can be communicated to the subject and/or a healthcare provider.

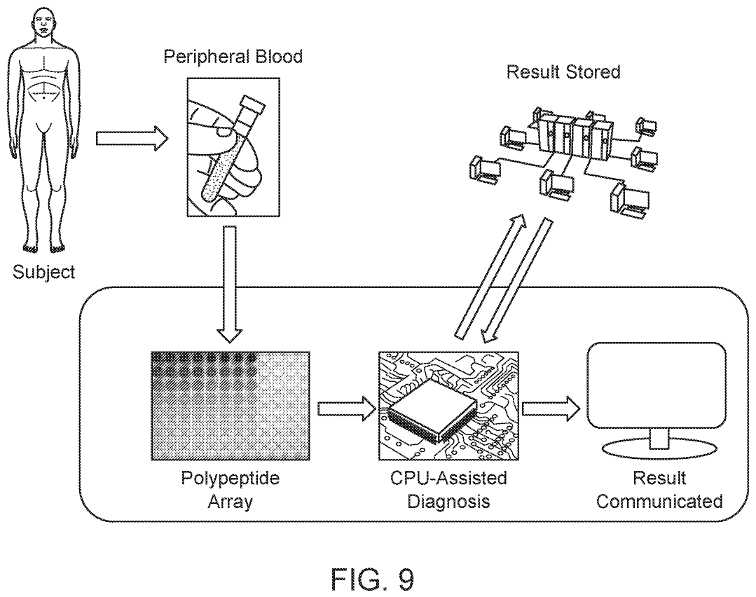

FIG. 9 provides an exemplary illustration of a computer implement workflow. Components in a sample from a subject indicative of stroke can bind to a protein probe and be detected in an assay as described herein. The assay output or binding intensity level can be fed into a system that can be used to distinguish between a stroke subject and a nonstroke subject, and/or between a hemorrhagic stroke and ischemic stroke subject. In some cases, the system can compare the binding intensity level to a reference as described herein. A result can be stored via local or cloud based storage for future use, and/or can be communicated to the subject and/or a healthcare provider.

In some aspects, a system can comprise software. A software can rely on structured computation, for example providing registration, segmentation and other functions, with the centrally-processed output made ready for downstream analysis.

In some aspects, the software would rely on unstructured computation, artificial intelligence or deep learning. In a variation of this aspect, the software would rely on unstructured computation, such that data could be iteratively. In a further variation of this aspect, the software can rely on unstructured computation, so-called “artificial intelligence” or “deep learning.” For example, a method described herein such as random forest can employ deep learning to generate gini impurity scores that can be used to parse out probes with improve predictive value.

The devices can comprise immunoassay devices for measuring profiles of polypeptides or proteins. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792. These devices and methods can utilize labeled probes in various stacked assays (e.g. sandwiches), competitive or non-competitive assay formats, to generate a signal that can be related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377. One skilled in the art can also recognize that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

The devices can comprise a filament-based diagnostic device. The filament-based diagnostic device can comprise a filament support which provides the opportunity to rapidly and efficiently move probes between different zones (e.g., chambers, such as the washing chamber or a reporting chamber) of an apparatus and still retain information about their location. It can also permit the use of very small volumes of various samples—as little as nanoliter volume reactions. The filament can be constructed so that the probes are arranged in an annular fashion, forming a probe band around the circumference of the filament. This can also permit bands to be deposited so as to achieve high linear density of probes on the filament.

The filament can be made of any of a number of different materials. Suitable materials include polystyrene, glass (e.g., fiber optic cores), nylon, carbon fiber, carbon nanotube, or other substrate derivatized with chemical moieties to impart desired surface structure (3-dimensional) and chemical activity. The filament can also be constructed to contain surface features such as pores, abrasions, invaginations, protrusions, or any other physical or chemical structures that increase effective surface area. These surface features can, in one aspect, provide for enhanced mixing of solutions as the filament passes through a solution-containing chamber, or increase the number and availability of probe molecules. The filament can also contain a probe identifier which allows the user to track large numbers of different probes on a single filament. The probe identifiers may be dyes, magnetic, radioactive, fluorescent, or chemiluminescent molecules. Alternatively, they may comprise various digital or analog tags.

### EXAMPLES

### Example 1: Study Overview

Peripheral whole blood was sampled from a cohort of acute ischemic stroke patients (n=19), hemorrhagic stroke patients (n=17), and acute stroke mimics (n=20) at emergency department admission. Circulating antibody profiles were generated from whole blood samples using protein array, and a two-step machine learning approach was subsequently used to select protein probes suitable for stroke diagnosis. First, random forest was used to rank all probes by importance in terms of their ability to discriminate between ischemic stroke, hemorrhagic stroke, and stroke mimic samples. Then recursive feature selection was used to identify the minimum number of top ranked probes which could provide optimal discriminatory performance. In order to evaluate the robustness of the analysis in terms of its ability to select optimally discriminatory probes, a permutation analysis was performed in which the diagnostic ability of the top ranked probes was compared to those selected at random.

### Example 2: Selection of Subjects

Acute ischemic stroke patients, hemorrhagic stroke patients, and acute stroke mimics were recruited at University of Cincinnati Medical Center (Cincinnati, Ohio). All ischemic stroke patients displayed definitive radiographic evidence of vascular ischemic pathology on MRI or CT according to the established criteria for diagnosis of acute ischemic cerebrovascular syndrome. All hemorrhagic stroke patients displayed definitive radiographic evidence of hemorrhagic pathology on MRI or CT. Patients admitted to the emergency department as suspected strokes based on the overt presentation of stroke-like symptoms, but receiving a definitive negative diagnosis for stroke upon neuroradiological imaging and clinical evaluation were identified as acute stroke mimics; the final discharge diagnoses of the stroke mimic group can be found in Table 2.

TABLE 2

| Primary emergency department complaint | Final diagnosis |
|---|---|
| 1. Altered mental status | Urinary tract infection |
| 2. Chest pain, weakness | Alcohol intoxication |
| 3. Vision problem, weakness | Bell's Palsy |
| 4. Altered mental status | Seizure |
| 5. Facial droop | Bell's Palsy |
| 6. Slurred speech | Bell's Palsy |
| 7. Altered mental status | Seizure |
| 8. Headache, vision problems | Complex migraine |
| 9. Altered mental status | Paresthesia |
| 10. Slurred speech, weakness | Complex migraine |
| 11. Extremity weakness, facial droop | Dehydration |
| 12. Altered mental status | Syncope |
| 13. Aphasia | Conversion disorder |
| 14. Altered mental status | Seizure |
| 15. Shortness of breath, altered | Adverse medication |
| mental status | reaction |
| 16. Aphasia, headache | Migraine |
| 17. Extremity weakness | Hypotension |
| 18. Altered mental status | Seizure |
| 19. Extremity weakness | Encephalopathy |
| 20. Headache, numbness | Complex migraine |

Patients were excluded if they received a non-definitive diagnosis or a diagnosis of transient ischemic attack, reported a prior hospitalization within 30 days, were under 18 years of age, or were admitted more than 12 hours post-symptom onset. Time from symptom onset was determined by the time the patient was last known to be free of neurological symptoms. Injury severity was determined according to the National Institutes of Health stroke scale (NUBS) at the time of blood draw. Demographic information was collected from either the subject or a significant other by a trained clinician. All procedures were approved by the institutional review board of University of Cincinnati Medical Center. Informed consent was obtained from all subjects or their authorized representatives prior to any study procedures.

Both ischemic and hemorrhagic stroke patients were significantly older than stroke mimics. Furthermore, ischemic and hemorrhagic stroke patients displayed a greater history of cardiovascular disease than stroke mimics, and a higher prevalence of cardiovascular disease risk factors, especially dyslipidemia. Ischemic stroke and hemorrhagic patients were relatively similar in terms of clinical and demographic characteristics, however the ischemic stroke group displayed a higher prevalence of dyslipidemia and contained a higher proportion of female subjects (Table 3).

TABLE 3

| | Stroke | | Ischemic | | Hemorrhagic | | | p values | | |
|---|---|---|---|---|---|---|---|---|---|---|
| | mimic | | stroke | | stroke | | Main | SM v | SM v | IS v |
| | (n = 20, SM) | | (n = 19, IS) | | (n = 17, HS) | | test | IS | HS | HS |
| ^{a}Age (mean ± SD) | 57.6 ± 13.9 | | 72.2 ± 14.2 | | 70.4 ± 12.4 | | 0.002* | 0.002* | 0.005* | 0.996 |
| ^{b}Female n (%) | 12 | (60) | 10 | (52.6) | 3 | (17.6) | 0.023* | 0.751 | 0.017* | 0.041* |
| ^{b}Caucasion n (%) | 13 | (65) | 14 | (73.7) | 13 | (76.5) | 0.756 | — | — | — |
| ^{b}African American n (%) | 7 | (35) | 5 | (26.3) | 4 | (23.5) | 0.756 | — | — | — |
| NIHSS (mean ± SD) | 1.5 ± 2 | | 11.3 ± 6.8 | | 12.3 ± 9.2 | | <0.001* | <0.001* | <0.001* | 0.683 |
| ^{a}Minutes to blood | 348.8 ± 155.8 | | 425.6 ± 236.6 | | 508.2 ± 158.7 | | 0.008* | 0.096 | <0.001* | 0.223 |
| draw (mean ± SD) | | | | | | | | | | |
| ^{b}History of stroke n (%) | 0 | (0) | 6 | (31.6) | 2 | (11.8) | 0.009* | 0.008* | 0.204 | 0.235 |
| ^{b}History of myocardial | 1 | (5) | 8 | (42.1) | 2 | (11.8) | 0.013* | 0.008* | 0.584 | 0.065* |
| infarction n (%) | | | | | | | | | | |
| ^{b}History of atrial | 3 | (15) | 8 | (42.1) | 2 | (11.8) | 0.069 | — | — | — |
| fibrillation n (%) | | | | | | | | | | |
| ^{b}Hypertension n (%) | 9 | (45) | 15 | (78.9) | 11 | (64.7) | 0.089 | — | — | — |
| ^{b}Dyslipidemia n (%) | 3 | (15) | 13 | (68.4) | 5 | (29.4) | 0.002* | 0.001* | 0.428 | 0.043* |
| ^{b}Diabetes n (%) | 5 | (25) | 4 | (21.1) | 3 | (17.6) | 0.920 | — | — | — |
| ^{b}Current smoker n (%) | 6 | (30) | 7 | (36.8) | 2 | (11.8) | 0.214 | — | — | — |

^{a}Means compared via one-way ANOVA with subsequent planned group-wise comparisons using two-sample two-tailed t-test;
^{b}Proportions compared via 2 × 3 Fisher's exact test with subsequent planned group-wise comparisons using 2 × 2 Fisher's exact test; SD, standard deviation.

### Example 3: Screening of Samples on Protein Array

Peripheral blood samples were obtained by venipuncture and collected via K₂EDTA vacutainer. EDTA-treated blood was aliquoted and stored immediately at −80° C. until analysis.

100 μL of whole blood was thawed and centrifuged to sediment hemocytes and debris. The supernatant was collected and diluted 1:1000 in phosphate buffered saline containing 0.5% bovine serum albumin and 0.05% Tween 20. Diluted soluble blood fractions were incubated on a silicon wafer array containing 125,000 unique protein probes. Following incubation, arrays were washed and incubated with AlexiFluor 647-conjugated pan anti IgG antibody. Afterwards, slides were again washed, dried, and imaged using a standard microarray scanner. Raw probe intensities were quantile normalized via the normalize.quantiles( ) function of the “preprocessCore” package for R (R project for statistical computing).

### Example 4: Random Forest Analysis

All statistics were performed using R version 3.4. The level of significance was established at 0.05 for all statistical testing. Fisher's exact test was used for comparison of dichotomous variables. Student t-test or oneway ANOVA was used for comparison of continuous variables where appropriate. Strength of correlations were assessed using Spearman's rho. Hierarchical clustering was performed using the “The performance of binary classifiers was assessed via receiver operator characteristic analysis (ROC) via the “pROC” package. The level of significance was established at 0.05 for all statistical testing. In the cases of multiple comparisons, p-values were adjusted using Benjamini-Hochberg method. Parameters of all statistical tests performed are outlined in detail within the figure legends.

Random forest models were generated via the “randomForest” package for R.13 Representative decision trees associated with random forest models were selected and visualized using the “reprtree” package.

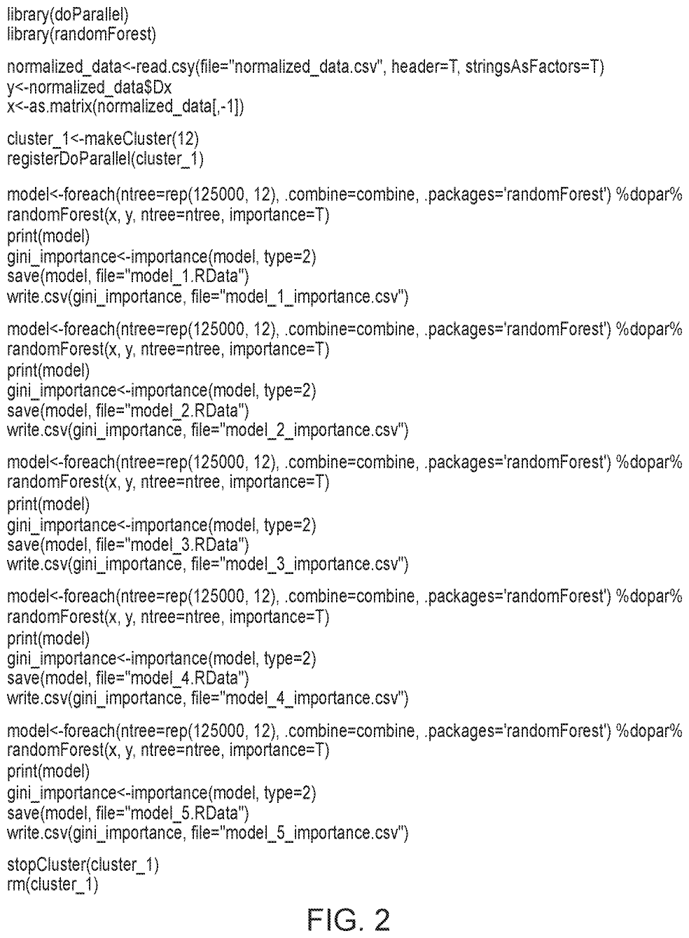

For ranking of probe importance, five replicate random forest models were built discriminating between ischemic stroke, hemorrhagic stroke, and stroke mimic samples using the log₂ transformed normalized intensity values of all 125,000 probes as input. 1.5 million decision trees were generated for each model, and probe importance was assessed in terms of node purity metrics, as quantified by mean decrease Gini coefficient. Probe importance was averaged across all five models and each probe was subsequently ranked. Script used for assessment of probe importance is depicted in FIG. 2.

For recursive feature section, successive combinations of the top ranked probes were evaluated for their ability to discriminate between experimental groups using random forest starting with the top probe and proceeding to the top two probes, the top three probes, the top four probes etc. Models were built using 50 times the number of decision trees relative to the number of input probes. For each random forest model, cross validation prediction probabilities were generated according to the vote distribution of the decision trees, yielding a predicted probability of ischemic stroke, hemorrhagic stroke, and stroke mimic for each sample. Hemorrhagic stroke and ischemic stroke prediction probabilities were combined to produce a total stroke prediction probability.

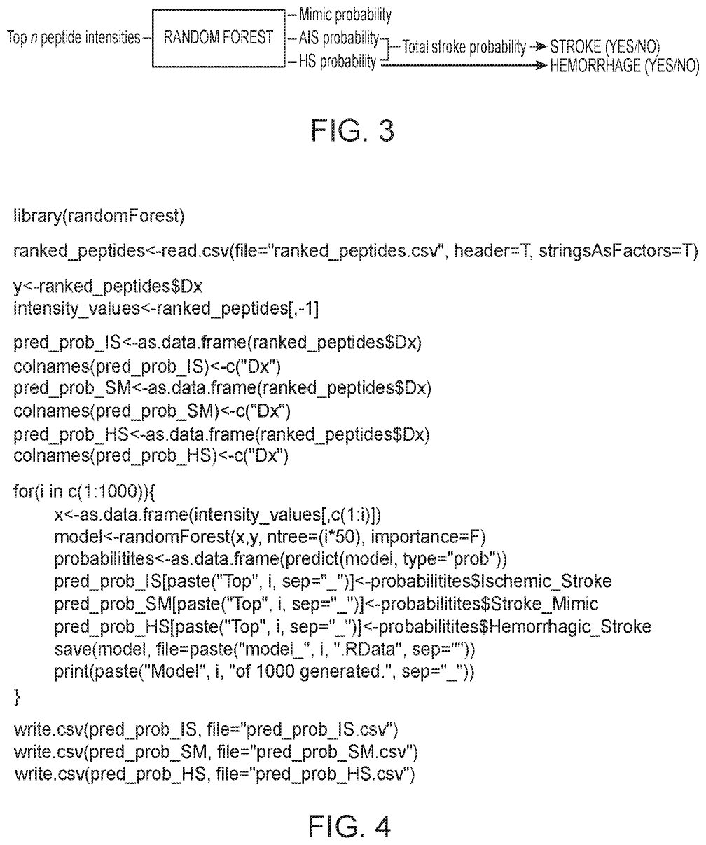

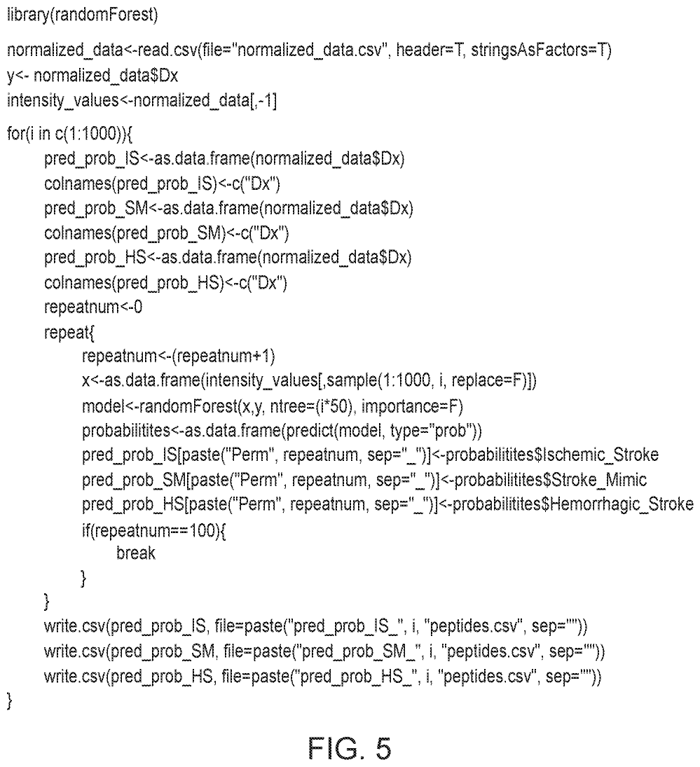

Total stroke prediction probability was used to classify samples as stroke/no stroke, and hemorrhagic stroke prediction probabilities were used directly to classify samples as hemorrhage/no hemorrhage (FIG. 3). Model classifications were then compared to true clinical diagnoses to assess accuracy. Script used to generate prediction probabilities for recursive feature selection is depicted in FIG. 4. For permutation analysis, 100 unique combinations of n probes were selected from the total probe pool, and the average diagnostic accuracy across the combinations was compared to that of the top n ranked probes. For example, the diagnostic accuracy of the top ten 10 probes was compared to the average diagnostic accuracy of 100 combinations of 10 randomly selected probes. Random probe combinations were generated using the R sample( ) function. Script used to generate prediction probabilities for permutation analysis is depicted in FIG. 5.

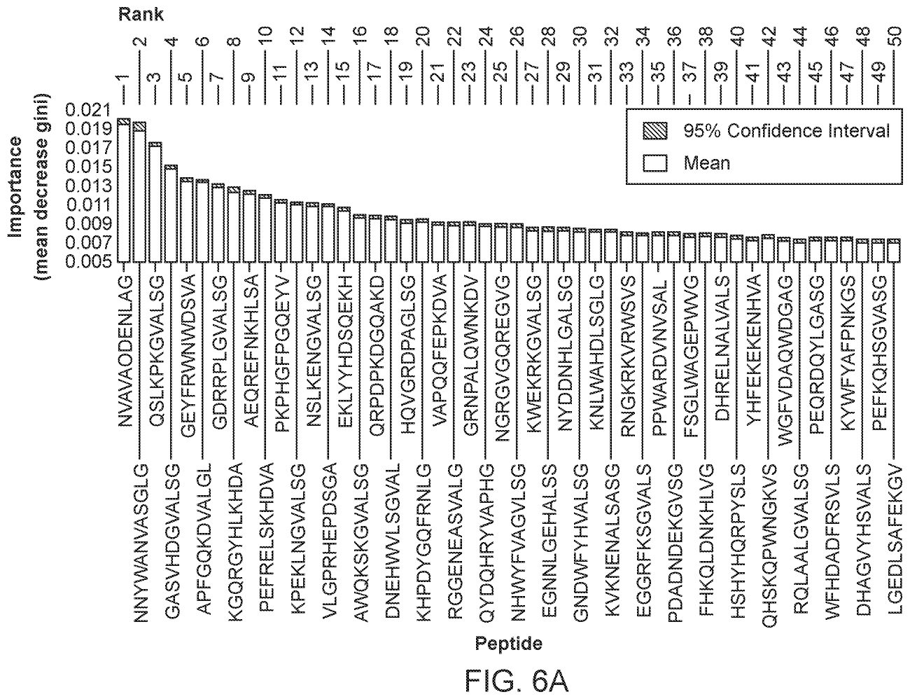

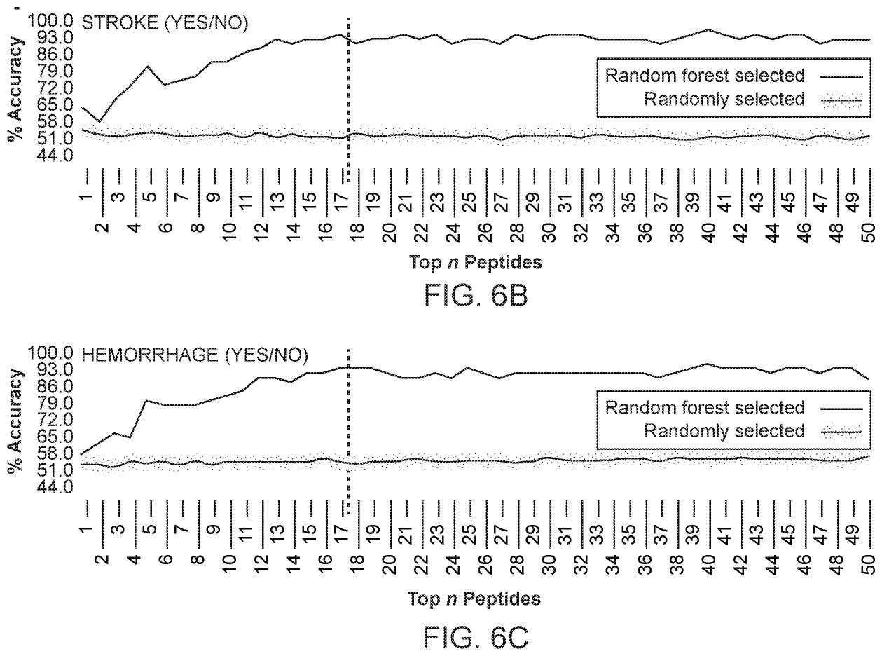

The top ranked protein probes, as determined by mean decrease Gini coefficient, are depicted in FIG. 6A. The combined ability of the top ranked probes to differentiate between stroke patents and stroke mimics in cross validation is depicted in FIG. 6B, while the combined ability of the top ranked probes to detect hemorrhage in cross validation is depicted in FIG. 6C. The top ranked probes displayed a markedly better discriminatory ability with regards to both stroke identification and hemorrhage detection relative to probes selected at random, suggesting that our analysis was successful in terms of selecting probes with robust diagnostic characteristics. Overall cross validation accuracy with regards to both identification of stroke and detection of hemorrhage appeared to plateau at 17 probes, and thus the model including the top ranked 17 probes was selected as the final model.

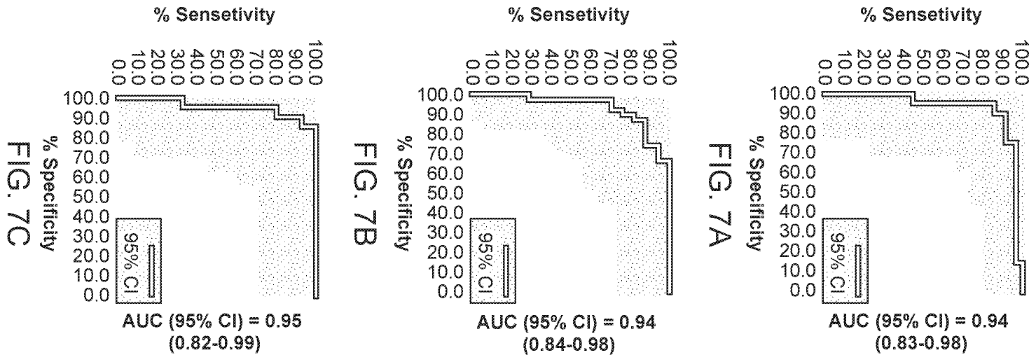

The top 17 probes used in combination were able to discriminate between stroke patients and stroke mimics with 91.7% sensitivity (95% CI=77.5-98.2%) and 90.0% specificity (95% CI=68.3-89.8%, FIG. 7A). The same 17 probes were able to detect hemorrhage with 88.2% sensitivity (95% CI=63.6-98.5%) and 87.1% specificity (95% CI=72.7-95.7%) when considering the total subject pool (FIG. 7B), and 93.3% sensitivity (95% CI=68.1-99.8%) and 90.0% specificity (95% CI=68.3-98.8%) when only considering patients first classified as stroke (FIG. 7C).

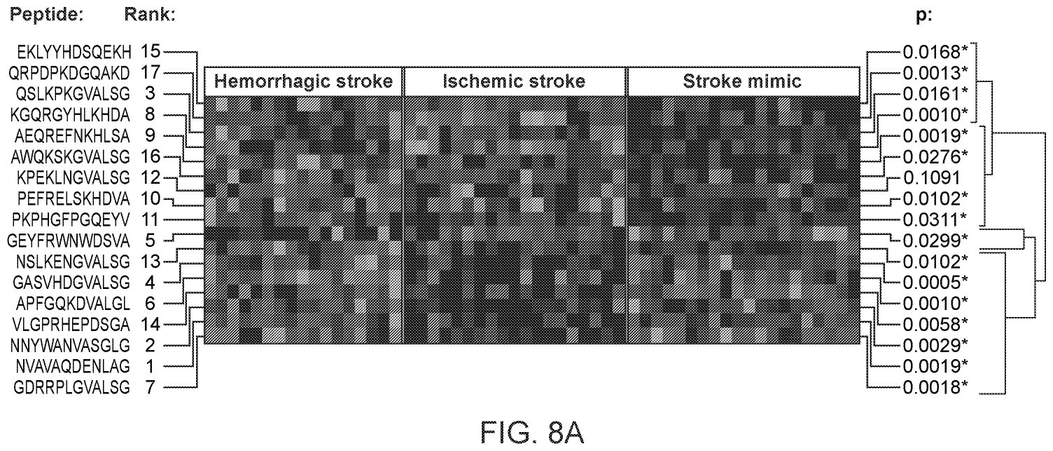

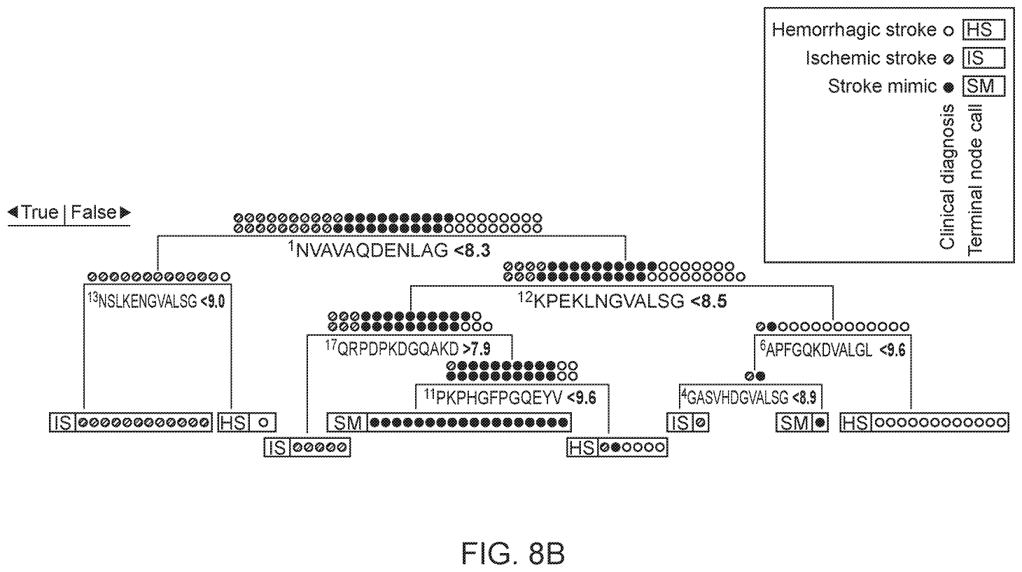

A comparison of the antibody binding intensity levels across the top 17 probes between ischemic stroke patients, hemorrhagic stroke patients, and stroke mimics is shown in FIG. 8A. Significant differences in antibody binding intensity levels were observed between groups with regards to each of the top 17 probes after controlling for multiple comparisons with the exception of one. Hierarchical clustering of the top 17 probes based on the correlational relationship between their antibody binding intensity levels produced three predominant clusters: one which displayed higher binding intensity levels in ischemic stroke patients relative to hemorrhagic stroke patients and stroke mimics, one which displayed lower binding intensity levels in ischemic stroke patients relative to hemorrhagic stroke patients and stroke mimics, and one which displayed higher binding intensity levels in ischemic and hemorrhagic stroke patients relative to stroke mimics.

Visualization of the final model's most representative decision tree revealed logical node spitting in terms of both the probe importance rankings generated in our probe selection paradigm, as well as the differential antibody binding intensity levels observed across the top ranked genes. For example, the root node of the tree was a split dependent on the binding intensity of the top ranked probe, NVAVAQDENLAG, which displayed lower binding intensity levels in ischemic stroke patients relative to hemorrhagic stroke patients and stroke mimics. Consistent with this pattern of differential binding, splitting the subject pool based on the root node criterion produced a relatively pure node comprised almost exclusively of ischemic stroke patients, and another node comprised predominantly of hemorrhagic stroke patients and stroke mimics (FIG. 8B).

The top 17 protein probes displayed a robust ability to both identify stroke and detect hemorrhage within a translationally relevant subject pool, indicating that diagnosis of stroke during triage using peripherally circulating antibody profiles is indeed feasible. The analysis shows that the circulating antibody pool can be altered in stroke. Due to the time it takes the adaptive immune system to produce fully-formed antibody responses, it is possible that the circulating antibody pool can be altered prior to the acute event as a result of immune changes preceding it. This surprising and unexpected result suggests that circulating antibody signatures could have diagnostic utility beyond triage, such as for identification of individuals at immediate risk of stroke prior to onset of symptoms. Such utility would be of great benefit in serial monitoring of high risk populations, such as individuals with known peripheral vascular disease or those recently experiencing transient ischemic attack.

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

What is claimed is:

1. A method comprising performing, using a computer processor, a random forest analysis on a first sample and a second sample, wherein the first sample and the second sample are associated with an array, wherein the first sample comprises a stroke patient biological sample and the second sample comprises a stroke mimic patient biological sample, wherein the array comprises at least one protein probe; wherein the random forest analysis comprises:
(a) comparing a binding intensity level of antibodies in the first sample with the at least one protein probe to a binding intensity level of antibodies in the second sample with the at least one protein probe; and
(b) generating a gini impurity score between the first sample and the second sample for the at least one protein probe.

2. The method of claim 1, further comprising performing multiple iterations of the random forest analysis, wherein the multiple iterations minimizes the gini impurity score between the first sample and the second sample for the at least one protein probe.

3. The method of claim 1 or 2, wherein the at least one protein probe comprises a plurality of protein probes; thereby generating a plurality of gini impurity scores between the first sample and the second sample for the plurality of protein probes.

4. The method of claim 3, further comprising performing, using the computer processor, a recursive analysis comprising:
(a) ranking the plurality of gini impurity scores;
(b) grouping a first set of the plurality of protein probes based on minimization of gini impurity scores between the first sample and the second sample to generate a first profile; and
(c) comparing the first profile to a second profile that comprises a second set of the plurality of protein probes, wherein the second set of the plurality of protein probes are not grouped based on minimization of gini impurity scores between the first sample and the second sample.

5. The method of any one of claims 1-4, wherein the stroke patient biological sample comprises a hemorrhagic stroke patient biological sample.

6. The method of any one of claims 1-4, wherein the stroke patient biological sample comprises an ischemic stroke patient biological sample.

7. The method of any one of claims 3-6, wherein the array comprises at least 100,000 protein probes.

8. A system for detecting stroke in a subject, the system comprising:
(a) a memory that stores executable instructions; and
(b) a computer processor that executes instructions to perform the method of any one of claims 1-7.

9. The system of claim 8, further comprising an integrated storage device.

10. A method comprising:
(a) contacting a sample with a synthetic protein;
(b) detecting a binding intensity level of antibodies in the sample with the synthetic protein; and
(c) comparing the binding intensity level to a reference, wherein the reference comprises a reference binding intensity level or a derivative thereof of antibodies in a stroke mimic sample with the synthetic protein.

11. The method of claim 10, wherein the sample was obtained from a subject.

12. The method of claim 11, wherein the subject has or is suspected of having a stroke.

13. The method of any one of claims 10-12, wherein the synthetic protein comprises an amino acid sequence at least 80% identical to any one of SEQ ID NO:1 to SEQ ID NO:50.

14. The method of claim 13, wherein the synthetic protein comprises an amino acid sequence at least 80% identical to SEQ ID NO: 1 or SEQ ID NO: 2.

15. The method of any one of claims 10-14, wherein the binding intensity level is at least about 1.5 fold higher than the reference binding intensity level.

16. The method of any one of claims 10-14, wherein the binding intensity level is at least about 1.5 fold lower than the reference binding intensity level.

17. The method of any one of claims 10-16, further comprising identifying the sample as a stroke sample or a stroke mimic sample.

18. The method of claim 17, wherein the identifying is with a sensitivity of at least 87% and a specificity of at least 87%.

19. The method of any one of claims 17-18, wherein the method comprises identifying the sample as a stroke sample.

20. The method of claim 19, wherein the identifying is with a sensitivity of at least 90%.

21. The method of claim 19 or 20, wherein the identifying is with a specificity of at least 90%.

22. The method of any one of claims 17-18, wherein the method comprises identifying the sample as a stroke mimic sample.

23. The method of claim 22, wherein the identifying is with a sensitivity of at least 90%.

24. The method of claim 22 or 23, wherein the identifying is with a specificity of at least 90%.

25. A method comprising:
(a) contacting a sample with one or more synthetic proteins, wherein the one or more synthetic proteins comprise an amino acid sequence at least 80% identical with any one of SEQ ID NO:1 to SEQ ID NO:50; and
(b) detecting a binding intensity level of antibodies in the sample with the one or more synthetic proteins.

26. The method of claim 25, wherein, the sample is obtained from a subject having a stroke or suspected of having a stroke.

27. The method of any one of claims 25-26, wherein the one or more synthetic proteins comprise an amino acid sequence at least 80% identical to any one of SEQ ID NO:1 to SEQ ID NO:17.

28. The method of any one of claims 25-27, wherein the one or more synthetic proteins comprise two or more amino acid sequence at least 80% identical to any one of SEQ ID NO:1 to SEQ ID NO:17.

29. The method of any one of claims 25-28, wherein the one or more synthetic proteins comprise three or more amino acid sequence at least 80% identical to any one of SEQ ID NO:1 to SEQ ID NO:17.

30. The method of any one of claims 25-29, wherein the one or more synthetic proteins comprise at least seventeen different synthetic proteins.

31. The method of any one of claims 25-30, wherein the one or more synthetic proteins comprises an amino acid sequence at least 80% identical to SEQ ID NO:1 or SEQ ID NO:2.

32. The method of any one of claims 25-30, wherein the one or more synthetic proteins comprises an amino acid sequence at least 95% identical to SEQ ID NO:1 or SEQ ID NO:2.

33. The method of any one of claims 25-32, further comprising comparing the binding intensity level to a reference.

34. The method of claim 33, wherein the reference comprises a reference binding intensity level or a derivative thereof of antibodies in an ischemic stroke sample, homographic stroke sample or stroke mimic sample with the one or more synthetic proteins.

35. The method of claim 34, wherein the binding intensity level is at least about 1.5 fold higher than the reference binding intensity level.

36. The method of claim 34, wherein the binding intensity level is at least about 1.5 fold lower than the reference binding intensity level.

37. The method of any one of claims 33-36, further comprising identifying the sample as an ischemic stroke sample, a hemorrhagic stroke sample or a stroke mimic sample.

38. The method of claim 37, wherein the identifying is with a sensitivity of at least 87% and a specificity of at least 87%.

39. The method of any one of claims 37-38, wherein the method comprises identifying the sample as an ischemic stroke sample.

40. The method of claim 39, wherein the identifying is with a sensitivity of at least 90%.

41. The method of claim 39 or 40, wherein the identifying is with a specificity of at least 90%.

42. The method of any one of claims 37-38, wherein the method comprises identifying the sample as a stroke mimic sample or a stroke sample.

43. The method of claim 42, wherein the identifying is with a sensitivity of at least 90%.

44. The method of claim 42 or 43, wherein the identifying is with a specificity of at least 90%.

45. The method of any one of claims 37-38, wherein the method comprises identifying the sample as a hemorrhagic stroke sample.

46. The method of claim 45, wherein the identifying is with a sensitivity of at least 87%.

47. The method of claim 45 or 46, wherein the identifying is with a specificity of at least 87%.

48. A kit comprising:
(a) a synthetic protein comprising an amino acid sequence at least 80% identical to any one of SEQ ID NO:1 to SEQ ID NO: 50; and
(b) a detecting reagent for detecting binding of an antibody with the synthetic protein.

49. The kit of claim 48, wherein the synthetic protein comprises an amino acid sequence at least 80% identical to any one of SEQ ID NO: 1 or SEQ ID NO: 2.

50. The kit of claim 48, wherein the synthetic protein comprises an amino acid at least 90% identical to any one of SEQ ID NO: 1 to SEQ ID NO: 50.

51. The kit of any one of claims 48-50, wherein the detecting regent comprises a secondary antibody.

52. The kit of claim 51, wherein the secondary antibody comprises a fluorophore.

53. A synthetic protein comprising an amino acid sequence at least 80% identical to any one of SEQ ID NO: 1 to SEQ ID NO: 50.

54. The synthetic protein of claim 53, comprising an amino acid sequence at least 95% identical to any one of SEQ ID NO:1 to SEQ ID NO:50.

55. The synthetic protein of any one of claims 53-54, comprising an amino acid sequence at least 95% identical to any one of SEQ ID NO:1 to SEQ ID NO:17.

56. The synthetic protein of one of claims 53-55, comprising an amino acid sequence at least 95% identical to SEQ ID NO: 1 or SEQ ID NO: 2.

57. The synthetic protein of any one of claims 53-56, wherein the synthetic protein is in an array.

58. A method comprising:
(a) contacting a sample with a synthetic protein, wherein the synthetic protein comprise an amino acid sequence at least 80% identical to any one of SEQ ID NO:1 to SEQ ID NO: 50;
(b) detecting a binding intensity level of antibodies in the sample with the synthetic protein; and
(c) comparing the binding intensity level to a reference.

59. The method of claim 58, wherein the sample was obtained from a subject.

60. The method of claim 59, wherein the subject has or is suspected of having a stroke.

61. The method of any one of claims 58-61, wherein the reference is a control.

62. The method of claim 61, wherein the reference is a non-stroke reference.

63. The method of any one of claims 58-62, wherein the reference is a reference binding intensity.

64. The method of any of preceding claim, wherein the sample comprises a cell-free sample.


SEQUENCE LISTING (50 sequences)

SEQ ID NO: 1 - protein, 12 aa, Artificial Sequence
  Description of Artificial Sequence Synthetic probe
NVAVAQDENLAG

SEQ ID NO: 2 - protein, 12 aa, Artificial Sequence
  Description of Artificial Sequence Synthetic probe
NNYWANVASGLG

SEQ ID NO: 3 - protein, 12 aa, Artificial Sequence
  Description of Artificial Sequence Synthetic probe
QSLKPKGVALSG

SEQ ID NO: 4 - protein, 12 aa, Artificial Sequence
  Description of Artificial Sequence Synthetic probe
GASVHDGVALSG

SEQ ID NO: 5 - protein, 12 aa, Artificial Sequence
  Description of Artificial Sequence Synthetic probe
GEYFRWNWDSVA

SEQ ID NO: 6 - protein, 12 aa, Artificial Sequence
  Description of Artificial Sequence Synthetic probe
APFGQKDVALGL

SEQ ID NO: 7 - protein, 12 aa, Artificial Sequence
  Description of Artificial Sequence Synthetic probe
GDRRPLGVALSG

SEQ ID NO: 8 - protein, 12 aa, Artificial Sequence
  Description of Artificial Sequence Synthetic probe
KGQRGYHLKHDA

SEQ ID NO: 9 - protein, 12 aa, Artificial Sequence
  Description of Artificial Sequence Synthetic probe
AEQREFNKHLSA

SEQ ID NO: 10 - protein, 12 aa, Artificial Sequence
  Description of Artificial Sequence Synthetic probe
PEFRELSKHDVA

SEQ ID NO: 11 - protein, 12 aa, Artificial Sequence
  Description of Artificial Sequence Synthetic probe
PKPHGFPGQEYV

SEQ ID NO: 12 - protein, 12 aa, Artificial Sequence
  Description of Artificial Sequence Synthetic probe
KPEKLNGVALSG

SEQ ID NO: 13 - protein, 12 aa, Artificial Sequence
  Description of Artificial Sequence Synthetic probe
NSLKENGVALSG

SEQ ID NO: 14 - protein, 12 aa, Artificial Sequence
  Description of Artificial Sequence Synthetic probe
VLGPRHEPDSGA

SEQ ID NO: 15 - protein, 12 aa, Artificial Sequence
  Description of Artificial Sequence Synthetic probe
EKLYYHDSQEKH

SEQ ID NO: 16 - protein, 12 aa, Artificial Sequence
  Description of Artificial Sequence Synthetic probe
AWQKSKGVALSG

SEQ ID NO: 17 - protein, 12 aa, Artificial Sequence
  Description of Artificial Sequence Synthetic probe
QRPDPKDGQAKD

SEQ ID NO: 18 - protein, 12 aa, Artificial Sequence
  Description of Artificial Sequence Synthetic probe
DNEHWVLSGVAL

SEQ ID NO: 19 - protein, 12 aa, Artificial Sequence
  Description of Artificial Sequence Synthetic probe
HQVGRDPAGLSG

SEQ ID NO: 20 - protein, 12 aa, Artificial Sequence
  Description of Artificial Sequence Synthetic probe
KHPDYGQFRNLG

SEQ ID NO: 21 - protein, 12 aa, Artificial Sequence
  Description of Artificial Sequence Synthetic probe
VAPQQFEPKDVA

SEQ ID NO: 22 - protein, 12 aa, Artificial Sequence
  Description of Artificial Sequence Synthetic probe
RGGENEASVALG

SEQ ID NO: 23 - protein, 12 aa, Artificial Sequence
  Description of Artificial Sequence Synthetic probe
GRNPALQWNKDV

SEQ ID NO: 24 - protein, 12 aa, Artificial Sequence
  Description of Artificial Sequence Synthetic probe
QYDQHRYVAPHG

SEQ ID NO: 25 - protein, 12 aa, Artificial Sequence
  Description of Artificial Sequence Synthetic probe
NGRGVGQREGVG

SEQ ID NO: 26 - protein, 12 aa, Artificial Sequence
  Description of Artificial Sequence Synthetic probe
NHWYFVAGVLSG

SEQ ID NO: 27 - protein, 12 aa, Artificial Sequence
  Description of Artificial Sequence Synthetic probe
KWEKRKGVALSG

SEQ ID NO: 28 - protein, 12 aa, Artificial Sequence
  Description of Artificial Sequence Synthetic probe
EGNNLGEHALSS

SEQ ID NO: 29 - protein, 12 aa, Artificial Sequence
  Description of Artificial Sequence Synthetic probe
NYDDNHLGALSG

SEQ ID NO: 30 - protein, 12 aa, Artificial Sequence
  Description of Artificial Sequence Synthetic probe
GNDWFYHVALSG

SEQ ID NO: 31 - protein, 12 aa, Artificial Sequence
  Description of Artificial Sequence Synthetic probe
KNLWAHDLSGLG

SEQ ID NO: 32 - protein, 12 aa, Artificial Sequence
  Description of Artificial Sequence Synthetic probe
KVKNENALSASG

SEQ ID NO: 33 - protein, 12 aa, Artificial Sequence
  Description of Artificial Sequence Synthetic probe
RNGKRKVRWSVS

SEQ ID NO: 34 - protein, 12 aa, Artificial Sequence
  Description of Artificial Sequence Synthetic probe
EGGRFKSGVALS

SEQ ID NO: 35 - protein, 12 aa, Artificial Sequence
  Description of Artificial Sequence Synthetic probe
PPWARDVNVSAL

SEQ ID NO: 36 - protein, 12 aa, Artificial Sequence
  Description of Artificial Sequence Synthetic probe
PDADNDEKGVSG

SEQ ID NO: 37 - protein, 12 aa, Artificial Sequence
  Description of Artificial Sequence Synthetic probe
FSGLWAGEPWVG

SEQ ID NO: 38 - protein, 12 aa, Artificial Sequence
  Description of Artificial Sequence Synthetic probe
FHKQLQNKHLVG

SEQ ID NO: 39 - protein, 12 aa, Artificial Sequence
  Description of Artificial Sequence Synthetic probe
DHRELNALVALS

SEQ ID NO: 40 - protein, 12 aa, Artificial Sequence
  Description of Artificial Sequence Synthetic probe
HSHYHQRPYSLS

SEQ ID NO: 41 - protein, 12 aa, Artificial Sequence
  Description of Artificial Sequence Synthetic probe
YHFEKEKENHVA

SEQ ID NO: 42 - protein, 12 aa, Artificial Sequence
  Description of Artificial Sequence Synthetic probe
QHSKQPWNGKVS

SEQ ID NO: 43 - protein, 12 aa, Artificial Sequence
  Description of Artificial Sequence Synthetic probe
WGFVDAQWDGAG

SEQ ID NO: 44 - protein, 12 aa, Artificial Sequence
  Description of Artificial Sequence Synthetic probe
RQLAALGVALSG

SEQ ID NO: 45 - protein, 12 aa, Artificial Sequence
  Description of Artificial Sequence Synthetic probe
PEQRDQYLGASG

SEQ ID NO: 46 - protein, 12 aa, Artificial Sequence
  Description of Artificial Sequence Synthetic probe
WFHDADFRSVLS

SEQ ID NO: 47 - protein, 12 aa, Artificial Sequence
  Description of Artificial Sequence Synthetic probe
KYWFYAFPNKGS

SEQ ID NO: 48 - protein, 12 aa, Artificial Sequence
  Description of Artificial Sequence Synthetic probe
DHAGVYHSVALS

SEQ ID NO: 49 - protein, 12 aa, Artificial Sequence
  Description of Artificial Sequence Synthetic probe
PEFKQHSGVASG

SEQ ID NO: 50 - protein, 12 aa, Artificial Sequence
  Description of Artificial Sequence Synthetic probe
LGEDLSAFEKGV